(19) United States
(12) Patent Application Publication
(10) Pub. No.: US 2014/0163075 A1
(43) Pub. Date: Jun. 12, 2014

(54) MODULATION OF THE UBIQUITIN-PROTEASOME SYSTEM (UPS)

(71) Applicants: Huib Ovaa, Amsterdam (NL); Celia R. Berkers, Amsterdam (NL); Yves Leestemaker, Amsterdam (NL); Karianne Shuurman, Amsterdam (NL); Annemieke De Jong, Amsterdam (NL)

(72) Inventors: Huib Ovaa, Amsterdam (NL); Celia R. Berkers, Amsterdam (NL); Yves Leestemaker, Amsterdam (NL); Karianne Shuurman, Amsterdam (NL); Annemieke De Jong, Amsterdam (NL)

(73) Assignee: NETHERLAND CANCER INSTITUTE, Amsterdam (NL)

(21) Appl. No.: 14/123,021

(22) PCT Filed: Jun. 1, 2012
(86) PCT No.: PCT/IB2012/001247

§ 371 Date: Feb. 14, 2014

Related U.S. Application Data
(60) Provisional application No. 61/591,361, filed on Jan. 27, 2012.
(60) Provisional application No. 61/491,938, filed on Jun. 1, 2011.

(51) Int. Cl. C07D 401/04; G01N 33/50

(52) U.S. Cl. CPC C07D 401/04; G01N 33/5076
USPC 514/341; 506/10; 546/274.1

(57) ABSTRACT

The invention relates to the field of 26S proteasome inhibition, activation and modulation and to identify compounds which activate 26S proteasome in live cells and a method of treating autoimmune diseases, cancer, inflammation and neurogenerative disorders by inhibition, activation and modulation of the 26S proteasome.

### CROSS REFERENCE TO RELATED APPLICATIONS

This application claims the benefit of priority of U.S. Provisional Application No. 60/491,938, filed Jun. 1, 2011 and U.S. Provisional Application No. 61/591,361, filed Jan. 27, 2012.

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The invention relates to the field of 20S and/or 26S and 20S proteasome activation and modulation and to identify compounds which activate 20S and/or 26S proteasome in live cells and a method of treating neurodegenerative diseases/disorders and diseases characterized by protein aggregation and/or protein deposition, autoimmune diseases, infection diseases, and inflammation, by activation and modulation of the 20S and/or 26S proteasome.

### BACKGROUND OF THE INVENTION

### 26S Proteasome Overview

The main cellular machinery for the degradation of intracellular proteins is the ubiquitin-proteasome system. Substrates tagged with a poly-ubiquitin tail are marked for degradation by the 26S proteasome. Because of the involvement of the 26S proteasome in many important cellular processes it has become a target of interest in the search of novel therapies for a wide variety of diseases. For instance, proteasome inhibitors have already proven themselves to be of great therapeutic value illustrated by the approval of Velcade® (bortezomib) for the treatment of multiple myeloma and mantle cell lymphoma.

In contrast to proteasome inhibition, proteasome activation is a relatively new field. Increasing the proteolytic capacity of cells could have potential therapeutic applications for the treatment of e.g. neurodegenerative diseases. The identification of proteasome modulators and the study of their regulating dynamics will contribute to the general knowledge about proteasome activity and assist in the development of new therapies for a variety of diseases.

In eukaryotes, proteins are constantly synthesized and degraded [2]. The 26S configuration of the proteasome is responsible for the degradation of proteins into short peptide fragments. Because of the enormous destructive potential of the 26S proteasome, both its localization and activity in a cell is under tight control [4]. Only proteins that are marked with a covalently linked poly-ubiquitin tail of at least 4 mono-ubiquitin molecules [5] in length can enter the 26S proteasome and are subsequently degraded into short peptide fragments. Not all proteins require poly-ubiquitination before 26S proteasomal degradation. One notable exception is ornithine decarboxylase (ODC) which activates its degradation by the 26S proteasome via a direct interaction with a regulatory subunit [6].

The ATP dependent post-translational modification of target proteins with ubiquitin is carried out by the concerted cooperation of three classes of enzymes. These enzymes can generate different ubiquitin linkages, but only chains that link mono-ubiquitins via the lysine at position 48 of the ubiquitin sequence are involved in proteasomal degradation [3]. The first step in creating poly-ubiquitin chains is the activation of a mono-ubiquitin by the E1 (ubiquitin activating) enzyme. The activated ubiquitin is bound to one of several E2 (ubiquitin conjugating) enzymes. Finally, the ubiquitin is covalently attached to the target protein by an E3 (ubiquitin ligase) enzyme [6]. This process then repeats itself and multiple mono-ubiquitins are attached to one another in this way to form a poly-ubiquitin tail. The process of ubiquitination is schematically represented in FIG. 2.

Especially the E3 enzymes are responsible for the selectivity of the UPS, as each E3 enzyme can only modify a single protein or a subset of proteins. Additional specificity is achieved by the post-translational modifications of substrate proteins, including phosphorylation [3]. After the substrate protein is polyubiquitinated by the E3 enzymes, it will undergo degradation via the 26S proteasome (see FIG. 5).

The main components of the 26S proteasome are depicted in FIG. 1. A 20S core particle (20S CP) is capped on one or both ends by a 19S regulatory particle (19SRP) [7] which is further described below.

### The 20S Core Particle

Highly conserved [3], the 20S CP is present in all eukaryotes, while some prokaryotes posses homologs of the 20S CP particle [6]. The 20S CP is a large cylinder shaped complex of 28 subunits and has an approximate molecular weight of 700 kDa [7]. The subunits are arranged in 4 heptametrical stacked rings in an α7-β7-β7-α7 configuration, as depicted in FIG. 3. The two outer rings are made up of α-type subunits, whereas the inner two rings are composed of β-type subunits [3]. At each end of the cylindrical complex there is a narrow gated pore [8]. This gated pore and its regulation play an important role in the protection against unregulated protein degradation by the active sites of the 20S proteasome [3].

The proteasome belongs to the family of the N-terminal nucleophile hydrolyses. [9]. Three of the 13 subunits, termed β1, β2 and β5, have a free N-terminal threonine residue, which is responsible for the proteolytic activity of the proteasome [10]. As each mature 20S CP consists of two β-rings each catalytic subunit is present twice. The three classical catalytic activities of the proteasome are designated chymotrypsin-like (β5), caspase-like (β1) and trypsin-like (β2) [9]. These subunits display a rough preference and will cleave after hydrophobic amino acids, negatively charged amino acids or positively charged amino acids, respectively. In addition to these three constitutive β subunits, mammals have three additional so called immunoproteasome subunits designated β1i, β2i and β5i [11]. The expression of these immunosubunits can be induced by interferon-gamma and these subunits can replace the constitutive subunits in proteasomes [12]. Proteasomes carrying such subunits are termed immunoproteasomes as they take part in generating peptide fragments that are more suitable for the induction of a immune response than those generated by constitutive β subunits [13]. In addition, hybrid proteasome subtypes, in which both constitutive and immunoproteasome are incorporated have been described. The ratio of constitutive and immunoproteasome β subunits appears to be organ specific [14]. While it is believed that equal amounts of catalytically active subunits are incorporated in proteasomes, the ratio of the different proteasomal activities is not necessarily equal [14]. This finding indicates that proteasomal activities can be fine-tuned in a specific manner to adapt to changing cellular requirements.

While free 20S CP is catalytically fully functional, it is only involved in the degradation of small peptides but not in the degradation of ubiquitinated proteins [6]. The gate of the 20S pore is in a closed conformation in cells [6], and folded proteins are unable to reach the chamber where the catalytically active sites are located [15-18]. In order to participate in the degradation of ubiquitinated substrates a 20S CP has to associate with at least one 19S RP. Through interaction with both poly-ubiquitinated substrates and the 20S CP, the 19S RP will facilitate 20S CP gate opening allowing access of substrates to the degradation chamber [19].

### The 19S Regulatory Particle

The 19S RP is the main regulatory component of the 26S proteasome [20]. It is responsible for the recognition of poly-ubiquitinated substrates, which is the basis for selective protein degradation [21]. The 19S RP functions include the opening the gate of the 20S CP, unfolding of the target substrate, removal of the poly-ubiquitin tail and the translocation of the unfolded polypeptide chain into the 20S CP [6]

The 19S RP (FIG. 4) consists of at least 17 core subunits, depending on the organism [20] and can be structurally divided in a base region and a lid region. In mammals, six of these subunits are ATPases of the AAA-superfamily and are designated as Rpt [20]. The other subunits are designated as Rpn and do not posses ATPase activity. The whole process of ubiquitin dependent degradation is depicted in FIG. 5. The poly-ubiquitin tail of a target substrate is recognized by one of the subunits of the 19S RP. This Rpn10 subunit will bind the poly-ubiquitin tail [6] upon which subunits Rpn1 and Rpn2 bind the substrate [22]. Since folded proteins are too big to enter the 20S CP, the target protein first has to be unfolded [20]. This is done by the ATPases of the 19S RP, although the exact contribution of each individual subunit is currently not known. The Rpt2 and Rpt5 subunit of the 19S RP induce an allosteric change in the 20S core particle, resulting in gate opening [15, 16], discussed in further detail below. After gate opening the ubiquitin tail is removed from the substrate by 19S RP subunit Rpn11, as well as by other deubiquitinating enzymes (DUBS) that associate with the proteasome [21]. The unfolded polypeptide chain of the substrate is translocated into the 20S core particle by the 19S RP ATPase subunits [8].

In mammals, there is less 19S RP than 20S CP present in the cell, resulting in a pool of free 20S CP as well as 26S proteasome only capped with an 19S RP at one side [6]. If there are functional differences between these two proteasomal configurations, it is currently not known. It is clear however that ubiquitin dependent degradation of substrates by the 26S proteasome requires the association of a 20S CP with at least one 19S RP. Before these two subunits can associate they have to first assemble separately.

### Proteasome Regulation: Assembly of the 265 Proteasome

Increasing proteasome activity could contribute to the development of new therapies for a variety of diseases. For instance, in Huntington's disease the UPS is not functioning properly and overexpression of the PA28 cap has been shown to have beneficial effects [29]. Furthermore, a recent publication suggests that increasing the activity of the 26S proteasome could be beneficial in the treatment of Alzheimer's disease [55]. One way of increasing the total amount of proteolytic activity in the cell is by increasing the total amount of 26S proteasome. The assembly pathway of 20S CP is fairly well understood, but relatively little is known about the assembly of 19S RP [23]. Furthermore, while there is evidence that there is crosstalk between the assembly pathways of these two sub-complexes [3, 6, 7] many questions remain to be answered.

The assembly of the 20S CP is a complex operation as it is made up out of four rings formed by seven distinct subunits, which each subunit occupying a defined position. The 20S core particle consists of four ring-like structures, each containing 7 subunits (α₁₋₇ and β₁₋₇), forming a hollow cylinder [22, 75, 32]. While the α-rings form the outer axial channel, the β-rings are located in the middle of the complex, with the active catalytic sites facing inwards into the channel [75, 76]. The assembly therefore requires several dedicated chaperones. The formation is initiated by the formation of α-rings where the Proteasome Assembly Chaperone complexes PAC1-PAC2 and PAC3-PAC4 make sure that each subunit is inserted at the correct position [23]. When these chaperones are not present, the incorrect incorporation of subunits will eventually lead to less active or defective proteasomes [7]. The presence of each individual α-subunit is also essential for the proper functioning of the mature 20S CP. For most of the α-subunits their absence or removal will result in incomplete assembly of 205 CP particles.

The α-ring/chaperone complexes form a platform for the formation of half-proteasomes. On this platform the β subunits assemble in a specific order. The β2 subunit is the first subunit that enters and starts the assembly of the β-ring upon which the chaperone Ubiquitin Maturation Protein UMP1 is recruited. Subsequently, β3, β4, β1, β5 and β6 join the complex in this fixed sequence and a half proteasome complex is formed. After β7 enters the complex two of these halves can dimerize to form an immature 20S CP [6, 7, 23, 24]. (The catalytic b-subunits are associated with specific activities: chymotrypsin-like (β5), trypsin-like (β2), and caspase-like (β1) activity [22, 76, 77]. The 19S regulatory particle, often caps both ends of the 20S core particle. It is understood to be involved in protein unfolding, so that proteins tagged for degradation can be threaded through the gated channel for proteolytic processing [75].)

After dimerziation the 20S CP undergoes maturation by autocatalytic removal of the pro-peptides from the proteolytic β subunits, the processing of some α and β subunits and the degradation of the chaperones that are still associated at this point. [3, 7]

The formation of the immunoproteasome, induced by interferon-gamma, is somewhat different compared to that of constitutive proteasomes. The βi subunits enter the complex in a different order and the total assembly is about four times faster than for constitutive proteasome. This increase in assembly speed is most likely due to higher concentrations of the chaperone UMP1, which is also induced by interferon-gamma. The faster assembly of the immunoproteasome upon immune stress ensures a rapid expansion of the peptide cleavage repertoire of an infected cell [23].

To participate in the degradation of polyubiquitinated substrates, the 20S core particle must first associate with at least one 19S RP. As mentioned above, in contrast to the 20S CP, the assembly pathway of the 19S RP is not very well studied [20]. It has long been assumed that 19S RP was assembled independently of the 20S CP. However, recent evidence suggests that the 20S CP does influence the 19S RP's assembly and/or stability [23]. It has been suggested that the α-ring of the 20S CP can act as a starting point for the initiation of 19S base complex assembly. The current view is that base and lid sections of the 19S RP are formed separately and that the two parts associate with each other prior to binding to 20S CP [23].

In order to form the 26S proteasome, a single 20S CP subunit and one or two 19S RP subunits have to associate. This association is ATP dependent [6]. A protein that has been implicated in the assembly and stabilization of 26S proteasome is Ecm29, a protein that can bind both to the 20S CP and 19S RP [26]. Recent literature suggests evidence that the proteasome is tightly regulated and involved in a regulatory network [75, 79]. However, the exact mechanism of association between 20S CP and 19S RP remains an open question, although the involvement of post translational modifications such as phosphorylation appear to be involved, i.e. signal transduction pathways. Phosphorylation of other subunits such as 20CP α2 and 19S Rpt6 also appear to increase the stability of the whole complex while dephosphorylation of these subunits is linked to the dissociation of the 26S proteasome into the 20S CP and 19S RP [3, 27, 28], suggesting that compounds that modulate the phosphorylation status of the proteasome may be used to modulate its activity. The entire process of 26S proteasome assembly is schematically represented in FIGS. 6 and 7. In FIG. 7, an example is given how post translational modifications of subunits can influence the assembly of the 26S proteasome.

Besides the 19S RP (PA700), the 20S CP can be capped by two other regulatory complexes, PA28 and PA200. Proteasomes capped with these regulatory complexes participate in pathways not related to the ubiquitin-dependent protein degradation. Furthermore, hybrid proteasomes subpopulations which have different caps on either side of the 20S CP have been identified but the physiological relevance of these complexes is currently unknown. A brief overview of the three different caps and their respective functions is given in Table 1.

TABLE 1
Brief overview of the 3 different regulatory particles or “caps”.

| | | ATP- | |
|---|---|---|---|
| Cap | Synonyms | dependent | Function |
| PA28 | 11S, REG | No | Promotes degradation of short peptides, |
| | | | but not complete proteins. Binding to the |
| | | | 20S core particle induces conformational |
| | | | changes to open the 20S pore gate. |
| | | | Expression is induced by interferon |
| | | | gamma. PA28 plays a role in generating |
| | | | peptides that can be presented by MHC |
| | | | proteins. [3, 29-32] |
| PA200 | | No | Promotes degradation of short peptides, |
| | | | but not complete proteins. Binding to the |
| | | | 20S core particle opens the 20S pore gate. |
| | | | Implicated in DNA damage response [3] |
| PA700 | 19S, RP | Yes | Promotes degradation of ubiquitinated |
| | | | proteins. Binding to the 20S core particle |
| | | | induces conformational changes to open |
| | | | the 20S pore gate (explained below). |
| | | | Furthermore the 19S subunit is required |
| | | | for the recognition, binding, unfolding |
| | | | and translocation of the ubiquitinated |
| | | | substrate. [3, 32-34] |

### Regulation 26S Proteasome: Gate Opening

Even when fully assembled, 26S proteasome is abundantly present inside the cell, protein degradation can only take place if the appropriate signals are present [20]. The 19S RP is responsible for recognizing poly-ubiquitinated substrates and preparing them for degradation. One of the essential steps is the opening of the narrow pore that provides access to the interior of the 20S CP. The poly-ubiquitin tail plays an important role in the induction of gate opening. From recent work it appears that in the absence of such a tail, the 26S proteasome exists predominantly in a semi-activated state in which the opening of the gate is not fully stabilized [19]. When a substrate with a poly-ubiquitin tail of at least four mono-ubiquitins [5] binds to the 19S RP, allosteric changes occur that will lead to a conformation in which the gate is more stable [19]. These changes likely involve pulling up the N-terminal sequences of α subunits of the 20S CP. This opens the gate and creates a continuous channel into the proteasome [31]. Mainly the α3 subunit of the 20S CP is believed to be obstructing the pore of the 26S proteasome when it is not activated. When this physical obstacle is removed there is an unobstructed path to the proteasome's central catalytic chamber, which allows both substrate to enter and cleavage products to be released. [31, 35].

### Proteasome Regulation: Post Translational Modifications

The catalytic activity of the 26S proteasome may be affected by various environmental factors such as oxidative stress, pathological states or pharmaceutical agents as well as by fundamental cellular processes such as apoptosis, proliferation or differentiation [6]. Post translational modifications (PTMs) of both target substrates and proteasomal subunits may alter or inhibit the functioning of the 26S proteasome [3]. Proteasomal subunits, like many other proteins, can undergo modifications such as phosphorylation [3, 27], N-actylation and/or N-terminal propeptide processing, 4-hydroxy-2-nonenal alkylation, O-glycosylation, S-gluthationylation, N-myristolyation and oxidation of sulphur containing amino acid residues [3, 6]. Furthermore, the proteasome interacts with an ever growing list of proteasome interacting proteins [39], which may lead to altered stability of the 26S complex and change its proteolytic activity. The intricate interplay of all these pathways will eventually determine the level of 26S proteolytic activity in a given cell. It also allows for a very dynamic system of regulation in which the 26S proteasome abundance and activity can quickly be adjusted to meet changing cellular circumstances. An overview of the many ways in which proteasome activity is regulated is depicted in FIG. 8.

Proteasome Regulation: p38-MAPK Signal Transduction Pathway

Preliminary screening experiments using a haploid cell line suggested that the p38-MAPK signal transduction pathway might be involved in proteasomal regulation.

Mitogen-activated protein kinases (MAPKs) play a major role in signal-transduction pathways involved in pro-inflammatory (immune) responses, regulation of cell-differentiation and proliferation, as well as apoptosis [81]. In particular, the p38-MPAK pathway triggers cellular response to various stimuli, i.e. environmental stressors, cytokines, growth factors, UV radiation. However, since MAPKs are involved in inflammatory processes, such as in rheumatoid arthritis or asthma [80-82], advances them as a promising drug target [80, 29]. Therefore, we investigated the role of the p38 MAPK pathway by inhibiting it with various inhibitors and investigating the outcome by SDS-PAGE and FACS (fluorescence-activated cell sorting) Firstly, the dynamics of one of the screening hits, the compound PD169316, a potential p38-MAPK inhibitor, was investigated. A panel of other known p38-MAPK inhibitors was also tested to determine their effects on the proteasome (FIG. 8A).

### 26S Proteasome Activators: Small Molecules

Small molecule proteasome inhibitors are well studies and have proven to be of great therapeutic value, as illustrated by the approval of Velcade® (bortezomib) for the treatment of multiple myeloma and mantle cell lymphoma. In contrast to the development of proteasome inhibitors, drug-like small molecules that can increase or enhance proteasome activity are rare and not well studied [29] and developing activators has proven to be more challenging compared to inhibitors [22]. While ways to inhibit the proteasome are well known, ways to enhance the proteasome are not.

Although compounds that are reported to activate the proteasome in a cellular context are rare, a wide variety of compounds have been reported that increase the conversion of fluorogenic substrates by the 20S proteasome in vitro. A list of some of these compounds is provided in Table 2. Most of these compounds were tested on their ability to induce the cleavage of small fluorogenic peptides and not of complete proteins tagged with a poly-ubiquitin tail. The latter requires recognition and processing by the 26 S proteasome [48]. As results obtained in in vitro experiments using 20S proteasome may not be representative of the capacity of compounds to modulate the UPS in a cellular environment, it is not surprising that all but two of the compounds listed in Table 2 failed to either increase the total amount of proteasome or the rate of proteasome cleavage when tested in live cells. Importantly, a small molecule inhibitor of the proteasome-associated DUB USP14 has been reported to enhance substrate degradation by the proteasome in cells [56]. This finding serves as a proof of principle that small molecules can be able to increase the activity of the 26S proteasome in cells. However the reported increase in activity was not very high [56]. In addition, only a single compound out of a library of 63,000 compounds was identified as a proteasome activator, illustrating the need for more sensitive methods to measure the 26S proteasome activity in cells and for the identification of proteasome activators.

TABLE 2
Overview of compounds that are reported to activate the proteasome.
is a summary of the results in both purified proteasome/cell lysate and
in live cells. For recents of clarity only representative compounds are
mentioned here.

| | Results: purified | Results: | |
|---|---|---|---|
| Compound | proteasome/lysate | live cells | Ref. |
| SDS | Increases all 3 proteasome | Not tested | [57, 58] |
| | activities | | |
| Polylysine | Increases mainly | Not tested | [57] |
| | chymotrypsin-like activity | | |
| Polyarginine | Increases mainly | Not tested | |
| | chymotrypsin-like activity | | |
| Oleic acid | Increases all 3 proteasome | Not tested | [58, 59] |
| | activities | | |
| Linoleic acid | Increases all 3 proteasome | Not tested | |
| | activities | | |
| alpha linolenic | Increases all 3 proteasome | Not tested | |
| acid | activities | | |
| Synthetic peptidyl | Increases all 3 proteasome | Not tested | [60] |
| alcohols, esters, | activities | | |
| p-nitroanilides and | | | |
| nitriles | | | |
| Ceramides. | Increases mainly | Not tested | [61-63] |
| Lysophosphatidyl- | chymotrypsin-like activity | | |
| inositol | | | |
| Cardiolipin | | | |
| Arginine-rich | Enhances caspase-like | Not tested | [9] |
| histone H3 | activity | | |
| | Decreases chymotrypsin- | | |
| | like activity | | |
| Oleuropein | Increases all 3 proteasome | No effect | [58] |
| | activities | | |
| Betulinic acid | Increases chymotrypsin- | Increases | [64] |
| | like activity | chymotrypsin- | |
| | | like activity | |
| IU1 | Increases all 3 proteasome | Increases all 3 | [56] |
| (usp14 inhibitor) | activities | proteasome | |
| | | activities | |

### Proteasome Activity Assays

As mentioned above, the identification of compounds that increase proteasome activity is hindered by the lack of good assays. To appropriately assess the effect of compounds on the UPS, solid methods are required that accurately determine the total amount of 26S proteasome in cells as well as its activity. A commonly used method to measure proteasome activity is to make use of fluorogenic substrates [14]. In this type of experiment small peptides are linked to a 7-Amino-4-methylcoumarin (AMC) group. Upon cleavage of this group by proteases such as the proteasome the AMC group becomes fluorescent and this signal can be measured over time. When the proper controls are included, the speed of conversion can be derived from such data and interpreted as a measure of proteasome activity [65]. The advantage of this method is that each of the catalytic subunits can be measured separately by using a substrate-AMC conjugate that is preferentially cleaved by one of the three catalytic subunits. One disadvantage is that only the activity of individual subunits can be measured, and not the total proteasome activity, as not all subunits may equally contribute to the total proteasome activity. Furthermore this type of experiment is almost always performed with cell lysates or purified proteasome. Data from this type of experiments may therefore not be relevant in more complex environments such as whole cells or the situation in vivo. Using cell permeable versions of fluorogenic substrates can help to overcome some these limitations, but these are currently not available for all 3 of the catalytic activities of the proteasome [65].

Therefore, there still exists a need by those of skill in the art for compounds and compositions that can activate or modulate the 26S proteasome and a means for identifying said compounds.

### SUMMARY OF THE INVENTION

The invention provides methods for identifying compounds and related compositions comprising said compounds that increase 20S and/or 26 proteasome activity. The invention also provides a method for increasing the activity of the 20S and/or 26S proteasome. The invention also provides for the compounds and compositions for the modulation of the 20S/26S proteasome and a method of modulating the 20S/26S proteasome. The invention further provides for the compounds that can be used for the treatment of neurodegenerative diseases/disorders and diseases characterized by protein aggregation and/or protein deposition, autoimmune diseases, infection diseases and inflammation by the activation or modulation of the 20S and/or 26S proteasome.

In an embodiment, the invention provides a composition for increasing the activity of 20S and/or 26S proteasome above basal levels, which comprises of the compounds identified as being activators of the 20S and/or 26S proteasome selected from the group consisting of calcium channel modulators, cAMP inhibitors, antiandrogens, methylbenzonium salts, PD169316 and proflavine and a pharmaceutically acceptable carrier.

In another embodiment, the identified compounds are selected from the group consisting of methylbenzethonium, PD169316, proflavine, cyclosporin A, loperamide, metergoline, pimozide, Win 62,577, verapamil, cyproterone, dipyrimadole, DPCPX, fenofibrate, medroxyprogesterone, mifepristone, pimozide, cyproterone, mifepristone, medroxyprogesterone and structural analogs thereof.

In an embodiment, the present invention provides a method for increasing the activity of the 20S and/or 26S proteasome above basal levels by administering a therapeutically effective amount of a composition, such as a composition described above, for increasing the activity of 20S and/or 26S proteasome to a patient in need thereof. The method of increasing the activity may be via direct or indirect activation of the 20S and/or 265 proteasome.

Methods of the present invention may be used to treat a patient in need thereof. In certain embodiments, a patient in need thereof may be a patient suffering from a neurodegenerative disease/disorder, a disease characterized by protein aggregation and/or protein deposition, an autoimmune disease, an infectious disease, cancer or inflammation.

In certain embodiments, a neurodegenerative disease/disorder, may be a disease characterized by protein aggregation and/or protein deposition is selected from the group consisting of Alzheimer's disease, Parkinson's disease, amyotrophic lateral sclerosis (ALS), Huntington's disease, transmissible spongiform encephalopaties (TSEs), Creutzfeld-Jakob disease, systemic amyloidosis, prion based diseases and diseases caused by polyglutamine repeats.

In certain embodiments, an autoimmune disease may be selected from the group consisting of alopecia areata, ankylosing spondylitis, arthritis, antiphospholipid syndrome, autoimmune Addison's disease, autoimmune hemolytic anemia, autoimmune inner ear disease (also known as Meniers disease), autoimmune lymphoproliferative syndrome (ALPS), autoimmune thrombocytopenic purpura, autoimmune hemolytic anemia, autoimmune hepatitis, Bechet's disease, Crohn's disease, diabetes mellitus type 1, glomerulonephritis, Graves' disease, Guillain-Barre syndrome, inflammatory bowel disease, lupus nephritis, multiple sclerosis, myasthenis gravis, pemphigus, pemicous anemia, polyarteritis nodosa, polymyositis, primary billiary cirrhosis, psoriasis, Raynaud's Phenomenon, rheumatic fever, rheumatoid arthritis, scleroderma, Sjogren's syndrome, systemic lupus erythematosus (SLE), ulcerative colitis, vitiligo, and Wegener's granulamatosis.

In an embodiment, an infectious disease may be a disease selected from the group consisting of disease associated with defective antigen presentation via MHC molecules.

In certain embodiments, cancer may be selected from the group consisting of leukemia; carcinoma of bladder, breast, colon, kidney, liver, lung, ovary, pancreas, stomach, cervix, thyroid, prostate, head, neck and skin; hematopoietic tumors of lymphoid lineage, acute lyphocytic leukemia; B-cell lymphoma; Burkett's lymphoma; hematopoietic tumors of myeloid lineage, acute and chronic myelogenous leukemias and promyelocytic leukemia; tumors of mesenchymal origin, fibrosarcoma, rhabdomyasarcoma; melanoma; seminoma; teratocarcinoma; osteosarcoma; neuroblastoma and glioma.

In certain embodiments, inflammation may be selected from the group consisting of rheumatoid arthritis, spondyloathopathies, gouty arthritis, osteoarthritis, systemic lupus erythematosis, juvenile arthritis, bronchitis, bursitis, gastritis, inflammatory bowel disease, ulcerative colitis, acne vulgaris, asthma, autoimmune dieases, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

In some embodiments of the present invention, a patient to be treated via the compositions and methods described herein may be suffering from Alzheimer's disease, Parkinson's disease or a prion-induced disease.

The present invention also provides a method of identifying a compound that may be described as compound (a), which increases the activity of 20S and/or 26S proteasome above basal levels which comprises:
i. obtaining a compound (a) from a compound library or other source;
ii. obtaining a cell type which expresses constitutive proteasome and incubating the cell type with the compound (a);
iii. adding a fluorescent probe to a cell culture of the cell-type, which binds covalently to the catalytic subunits of the 2DS CP of the 26S proteasome to transform the catalytic subunits of the 20S or 26S proteasome into a 20S or 26S proteasome-fluorescent probe complex;
iv. measuring fluorescence (FL-1) of the 20S or 26S proteasome-fluorescent probe complex by flow cytometry and/or confocal microscopy and image analysis of a combination thereof and measuring the forward scatter, side scatter to create a score for proteasome activity and converting the score to an FL-1 log 2 ratio relative to the average of untreated cells,
   v. identifying compound (a) which have a FL-1 log 2 ratio greater than 1.00;
   vi. validating the identified compound (a) in step v. by:
   a. repeating steps i.-iv.; and
   b. repeating steps i.-iii., followed by lysing the cells to form a cell lysate which is resolved by SDS-PAGE and subsequently analyzed by fluorescent scanning of the resulting gel;
vii. identifying compound (a) which still have a FL-1 log 2 ratio greater than 1.00 after step vi. a. and have bands that show dose-dependent increased fluorescence for the β2 and β5 subunits of the 20S or 26S proteasome after step vi. b.

The present invention provides pharmaceutical compositions comprising one or more compounds (a) identified as being activators of the 20S and/or 26S proteasome by the screening process described in the foregoing.

In certain embodiments, compound (a):
i, has a side scatter (SSC) less than average of DMSO+3× Standard Deviation (SD); and/or has a number of events greater than DMSO−3×SD; and
ii. has FL-2 and/or FL-3 less than average of DMSO+3×SD.

The present invention provides a method for increasing the activity of the 20S and/or 26S proteasome above basal levels by administering a therapeutically effective amount of a compound (a) identified by screening to a patient in need thereof. The method of increasing the activity may be by the direct or indirect activation of the 20S and/or 26S proteasome.

In one embodiment, the present invention provides a compound of formula I:

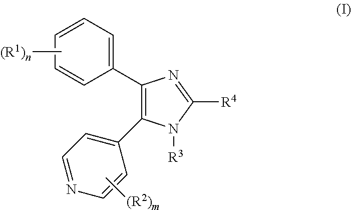

wherein,
R¹ is halogen, hydroxyl, amino, C₁-C₄ alkyl;
R² is halogen, hydroxyl, amino, C₁-C₄ alkyl;
R³ is hydrogen or C₁-C₄ alkyl;
R⁴ is phenyl or phenyl substituted with halogen, hydroxyl, amino, C₁-C₄ alkyl, nitro, sulfinyl, C₁-C₄ alkylsulfinyl; sulfonyl or C₁-C₄ alkylsulfonyl; or
R³ and R⁴ together form a 5-6 membered ring with at least one additional heteroatom selected from the group consisting of O, N and S;
n is 0-5; and
m is 0-4.

In another embodiment of the compound of formula (I):

R¹ is halogen;
R² is halogen;
R³ is hydrogen or CH₃;
R⁴ is phenyl substituted with halogen, hydroxyl, amino, C₁-C₄ alkyl, nitro, methylsulfonyl or methylsulfonyl; or
R³ and R⁴ together form a 5-6 membered ring with at least one additional heteroatom selected from the group consisting of O, N and S;
n is 0-2; and
m is 0-1.

In another embodiment of the compound of formula (I):

### R¹ is F;

R³ is hydrogen or CH₃;
R⁴ is phenyl substituted with F, hydroxyl, amino, CH₃, nitro, or methylsulfinyl; or
R³ and R⁴ together form a 5 membered ring with S as one additional heteroatom;
n is 0-1; and
m is 0.

In another embodiment the compound of formula (I) is:

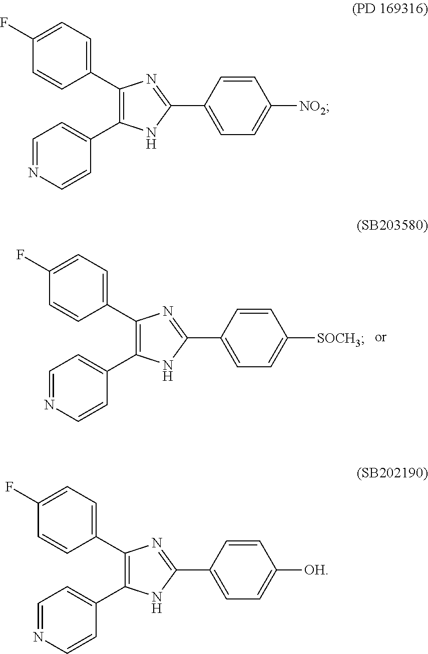

### LIST OF ABBREVIATIONS

### 19S RP 19S Regulatory Particle

### 20S CP 20S Core Particle

26S Proteasome One 20S CP capped on one or both sides by 19S RP

### ATP Adenosine Tri-Phosphate

AMC 7-amino-4-methylcoumarin
DUB Deubiquitinating enzyme

### FACS Fluoroescence Activated Cell Sorting

### HTS High Throughput Screening

### JHCCL Johns Hopkins Clinical Compound Library

### LOPAC Library of Pharmacologically Active Compounds

### MHC Major Histocompatibility Complex

### PAC Proteasome Assembly Chaperone

### PIP Proteasome Interacting Protein

ODC Ornithine decarboxylase
kDa kilodalton

### Poly-Ub Poly-Ubiquitin

### PTM Post Translational Modification

siRNA small interfering RNA
shRNA short hairpin RNA

### UPS Ubiquitin-Proteasome System

The term “modulation/regulation” or “modulate/regulate” as used in this specification describes controlling the activity of the 20S and/or 26S proteasome by selectively or continuously activating the 20S and/or 26S proteasome with a compound/composition that increases/decreases 20S and/or 26S proteasome activity or that increases/decreases the amount of cellular 20S and/or 26S proteasome and thereby increases/decreases the total amount of proteasome activity. The modulation may also be applied during the treatment of a disease or condition.

The phrase “direct activation of 26S proteasome” as used in this specification describes increasing the activity of 20S and/or 26S proteasome or increasing the amount of (active) cellular 20S and/or 26S proteasome above basal levels via direct interaction with the 20S or 26S proteasome, thereby increasing the total amount of proteasome activity in the cell via a direct interaction with the 20S and/or 26S proteasome. Direct activation can also apply to administering a therapeutically effective amount of a 20S and/or 26S proteasome activating compound to a patient.

The phrase “indirect activation of 20S and/or 26S proteasome” as used in this specification describes all ways of increasing proteasome activity other than via ‘direct activation’ and describes increasing the activity of 20S and/or 26S proteasome or increasing the amount of (active) cellular 20S and/or 26S proteasome and thereby increasing the total amount of proteasome activity in the cell via a cellular environment/another component in the cellular environment. Indirect activation can also apply to administering a therapeutically effective amount of a 20S and/or 26S proteasome activating compound to a patient.

The term “disease” describes any deviation from or interruption of the normal structure or function of any body part, organ, or system that is manifested by a characteristic set of symptoms and signs whose etiology, pathology, and prognosis may be known or unknown. (Dorland's Pocket Medical Dictionary, 24^{th} Edition, pg. 179, (1989)).

The term “disorder” describes a derangement or abnormality of function; a morbid physical or mental state. (Dorland's Pocket Medical Dictionary, 24^{th} Edition, pg. 185, (1989)).

The term “analog” describes a chemical compound having a structure similar to that of another but differing from it in respect to a certain component (Dorland's Pocket Medical Dictionary, 24^{th} Edition, pg. 26, (1989)).

The term “derivative” describes a chemical substance derived from another substance either directly or by modification or partial substitution (Dorland's Pocket Medical Dictionary, 24^{th} Edition, pg. 26, (1989)).

The term “prodrug” describes a compound that, on administration, must undergo chemical conversion by metabolic processes before becoming an active pharmacological agent; a precursor of a drug (Dorland's Pocket Medical Dictionary, 24^{th} Edition, pg. 490, (1989)).

These and other embodiments are disclosed or are obvious from and encompassed by, the following Detailed Description.

### BRIEF DESCRIPTION OF THE DRAWINGS

FIG. 1: Canonical representation of the 26S proteasome. The 20S core particle is capped at both sides by 19S regulatory particles. The poly-ubiquitin chain of the target substrate is recognized by the regulatory particle, which binds the target protein, removes the ubiquitin chain, unfolds the target protein and translocates the protein into the proteolytic cavity where it is cleaved in short peptide fragments that leave the proteasome. PA700=19S RP. Adapted from McNaught, 2001 [1].

FIG. 2: Poly-ubiquitination of substrate protein by E1, E2 and E3 enzymes. Ubiquitin is activated by ubiquitin activating enzyme E1 and translocated to ubiquitin conjugating enzyme E2. In the last stage, ubiquitin ligase E3 conjugates ubiquitin to the target protein. By repetition of this process multiple ubiquitin molecules are attached to the target protein and a poly-ubiquitin chain is formed. Adapted from Sorokin, 2009 [6].

FIG. 3: Composition and dimensions of the 20S CP. The 20S CP consists of 4 heptametrical stacked rings. The outer 2 rings consist of α subunits (red), whereas the 2 inner rings consist of β subunits (blue). At each end, a narrow, gated pore provides access to the interior.

FIG. 4: Schematic representation of the architecture of the 19S RP. The complex can be subdivided in a “base” region and a “lid” region. Adapted from Sorokin, 2009 [6].

FIG. 5: Simplified model of ubiquitin-dependent degradation of proteins by the proteasome. The target is first tagged with a poly-ubiquitin tail (step 1). This tag is recognized by the 19S regulatory particle (step 2). After recognition the RP binds the substrate (step 3) and unfolds it (step 4). The gate of the 20S core particle is opened (step 5), and the poly-ubiquitin tail is removed from the substrate (step 6). The substrate polypeptide chain is threaded into the 20S CP, where the peptides are hydrolyzed by the 20S CP catalytic subunits. Adapted from Sorokin, 2009 [6].

FIG. 6: Schematic model of the proposed 26S proteasome assembly in eukaryotes. Two chaperone complexes, PAC1-PAC2 and PAC3-PAC4, assist in assembling subunits α1-α7 into heptametrical rings, on which the β subunits can assemble. 32 enters first, followed by another chaperone UMP1. After β3 and β4 have entered the complex PAC3-PAC4 is removed. β5, β6 and β1 join in the complex forming “half-proteasomes” only lacking P7. Upon binding of β7 the two halves can dimerize. The proteasome is activated by autocatalytic cleavage of its β subunits and subsequently degrades the chaperones PAC1-PAC2 and UMP1. PA200 is replaced by 19S RP as a cap. Adapted from Marques, 2009 [23].

FIG. 7: Hypothetical model on the regulation of 26S proteasome assembly. Auto-phosphorylation activity of the dissociated 19S RP is activated, or the phosphorylation site is exposed upon dissociation from the 26S proteasome. When the p45 subunit of the 19S RP is phosphorylated, the 19S RP is capable of associating with the 20S CP. This results in assembly of the 26S proteasome. The phosphorylated p45/Rpt6 directly interacts with the α2 subunit of the 20S CP. Image and description from Satoh, 2001 [27].

FIG. 8: Proteasome plasticity. Alternative incorporation of caps, subunits and post translational modifications regulate proteasome activity, specificity and localization according to cellular needs. Possible outcomes of such modifications are e.g. increased stability of the 26S proteasome or dissociation of the 26S proteasome into 20S and 19S sub complexes resulting in a reduction of proteolysis. Proteasome disassembly also occurs after subunit cleavage by caspases or when ATP is no longer present. PIPs are Proteasome Interacting Proteins. Image from Glickman, 2005 [36].

FIG. 8A: Schematic diagram of experimental setup to determine effects of p38-MAPK inhibitors on the proteasome.

FIG. 9: A schematic representation and the structure of the Me₄BodipyFL-Ahx₃Leu₃VS proteasome activity probe. The probe contains a reactive vinylsulfone part (VS), coupled to three leucine residues (L₃), a spacer consisting of three aminohexanoic acid moieties (Ahx₃) and a Me-₄BodipyFL fluorophore. Image adapted from Berkers 2005 [51], description adapted from Berkers, 2007 [14]

FIG. 10: Overview of the workflow used to screen two compound libraries for proteasome activating compounds using a FACS based activity assay. A cell suspension containing 2*10⁵ cells was prepared (1). Using a Wellmate microplate dispenser the cell solution was transferred to black 384 wells plates (2). The plates were then incubated for 24 hours (3). Using a Hamilton liquid handling workstation the compounds were added to the plates (4). Cells were incubated for 16 hours after which proteasome activity probe was added. After two hours of incubation with probe the cells were fixed and prepared for FACS analysis (6). One by one the plates were brought to a FACS Calibur equipped with a HTS unit. Subsequently, the plates were measured and the data were analyzed and evaluated.

FIG. 11: Top left: evaluation of DMSO controls and exclusion of aberrant wells. Top right: Calculations of the average and standard deviation of four parameters from the DMSO samples. “Max” and“min” refer to the average of DMSO±3× the standard deviation Bottom: Example analysis of four fictional compounds C1-C4. C1 has a value <0 and is therefore identified as a proteasome inhibitor. C2 and C3 have both values >0, however, while both C2 and C3 have a FL-1 log 2 ratio >1, C3 is excluded as a proteasome activator because of high SSC and low # of events. C4 has a positive FL-1 log 2 ratio, but scores lower than the cutoff value of FL-1 log 2 ratio=1, and is therefore not classified as a proteasome activator.

FIG. 12: Representative graphs from inhibition/activation experiments using the fluorescent Me₄BodipyFL-Ahx₃Leu₃VS proteasome activity probe to monitor proteasome activity. Top graph shows differences in signal between labeled (yellow) and unlabeled (black) MelJuSo wild type cells. Proteasome inhibition by MG132 (red) reduces the signal compared to untreated cells. Bottom graph shows the dose-dependent increase of the fluorescent signal upon addition of the proteasome activator Win 62,577.

FIG. 13: Validation of compounds from the LOPAC and JHCCL libraries using a FACS-based assay. MelJuSo wilt type cells were incubated with 5 μM compound for 16 hours. Prior to FACS analysis, the cells were stained with the proteasome activity probe, followed by fixation. FL-1 scores were converted to FL-1 log 2 ratios. Three compounds with a FL-1 log 2 ratio >1.0 failed validation.

Results from FACS experiment, in which activators found in this screen are compared to the USP14 inhibitor reported to increase proteasome activity [21]. MelJuSo wild type cells were incubated for 16 hours with 5 μM of compound, stained with proteasome activity probe, harvested and measured as described. FL-1 values were converted into FL-1 log 2 ratios. Empty columns represent compounds identified during this study and the patterned column the USP14 inhibitor. The experiment was performed in triplicate and error bars represent SD.

FIG. 14: FL-1 log 2 ratios of LOPAC compounds determined by FACS analysis. MelJuSo wild type cells were incubated for 16 hours with increasing concentrations of compound, stained with proteasome activity probe, harvested and measured as described. FL-1 values were converted into FL-1 log 2 ratios. For all compounds tested there is a concentration dependent increase in proteasome activation visible.

FL-1 log 2 ratios of all JHCCL compounds initially identified in the screen determined by FACS analysis. MelJuSo wild type cells were incubated for 16 hours with increasing concentrations of compound, stained with proteasome activity probe, harvested and measured as described. FL-1 values were converted into FL-1 log 2 ratios. For all compounds tested there is a concentration dependent increase in proteasome activation visible. Benztropin, medroxyprogesterone and escitalopram failed to meet the FL-1 log 2>1 criteria and were not taken along for further experiments.

FIG. 14A: SDS-PAGE gel image showing the activity of the labeled proteasome subunits for increasing concentrations of the compound PD 169316 in MEL-JUSO cells. The β2 and β5-subunits exhibit the strongest activation effect at a concentration of 3.0 and 10.0 μM

FIG. 14B: SDS-PAGE gel image showing inhibition of proteasome subunits in MEL-JUSO cells following incubation with PD169316, PD98059, SB202150, SB203580, and SKF 86002 (1 μM and 5 μM). The bands corresponding to the β2 and β5-subunits are most pronounced for SB202150 and PD98059 (5 μM).

FIG. 14C: Histogram showing the logarithmic fluorescence signal intensity with increasing PD16913 concentrations. The experiment was performed once in duplicate (n=1); the error bars correspond to the standard deviation (SD).

FIG. 14D: Fluorogenic substrate assay using KBM7 cells incubated with 3 different PD169316 concentrations. The plot shows the pronounced activating effects of the compound at 1 and 5 μM.

FIG. 15: FL-1 log 2 ratios of compounds determined by FACS analysis. MelJuSo wild type cells were incubated with different concentrations of compound for different time points. Samples were measured and FL-1 values converted into FL-1 log 2 ratios. For most LOPAC and JHCLL compounds tested, the proteasome activating potential appears to be concentration, but not time dependent.

For DPCPX, the proteasome activation appears to be both concentration and time dependent. All concentrations are in μM. (LOPAC compounds PD169316, Win 62,577, dipyrimadole, loperamide, pimozide, metergoline, verapamil and DPCPX; JHCLL compounds cyclosporine A, proflavine, cyproterone, mifepristone, fenofibrate and methylbenzethonium)

FIG. 16: Results from SDS-PAGE experiment. Cells were exposed to the same concentrations of compound, lysed, resolved and measured as described previously. Again a concentration dependent increase in signal is observed. NT=No Treatment and MG=MG132.

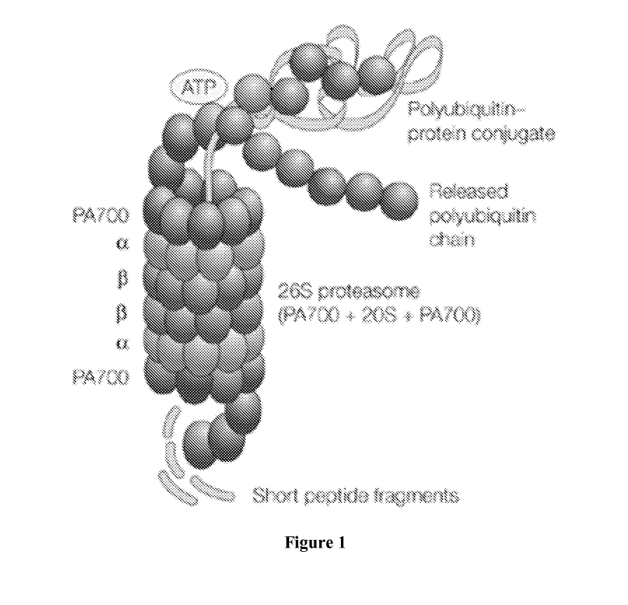

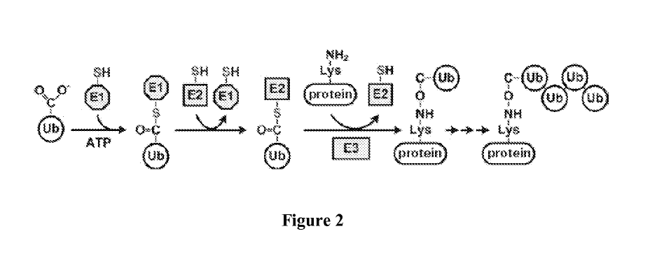

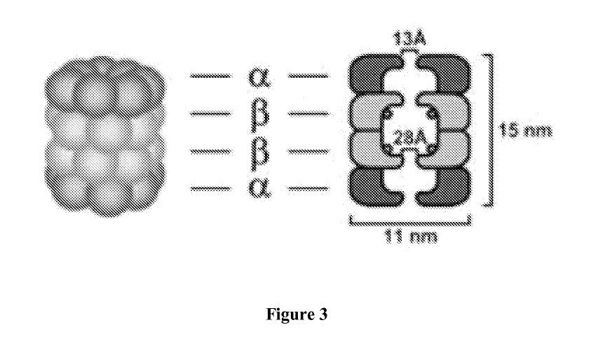

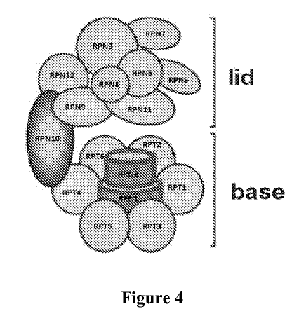

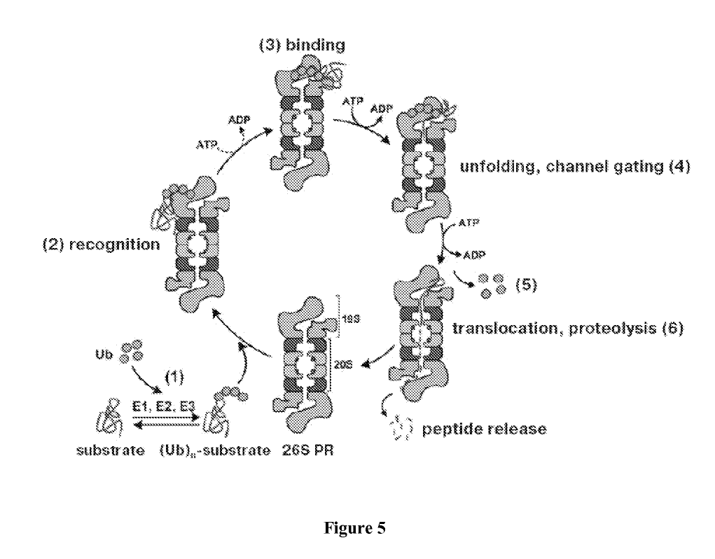

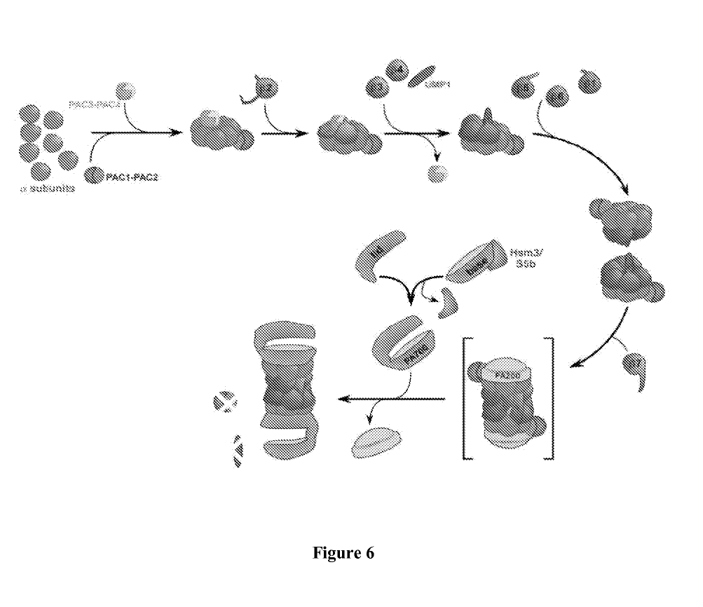

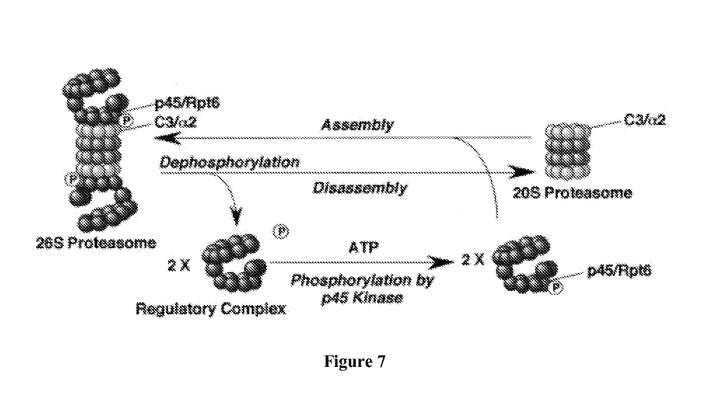

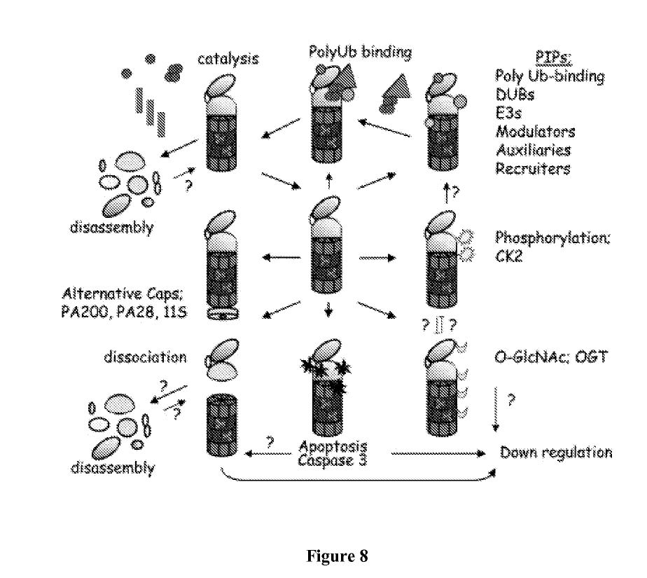

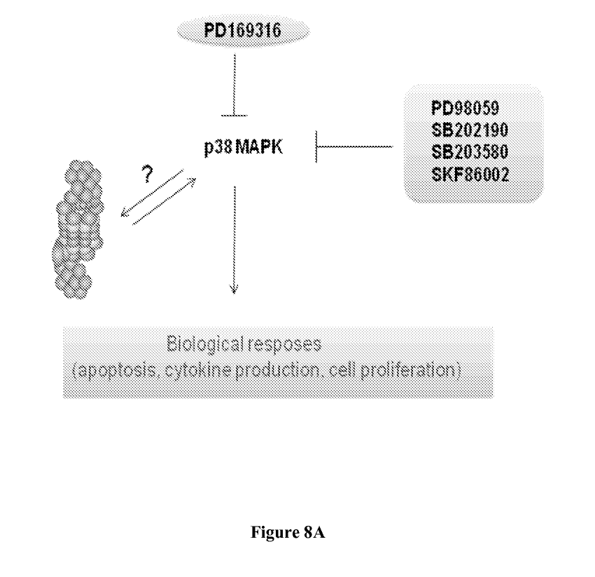

Fluorescent scan of SDS-PAGE analysis of the effect of the LOPAC compounds on proteasome activity. MelJuSo wild type cells were incubated with increasing concentrations of compound, stained with proteasome activity probe, lysed, resolved and measured as described in material and methods. For all compounds a concentration dependent increase in signal is observed.

Fluorescent scan of SDS-PAGE analysis of the effect of the JHCCL compounds on proteasome activity. MelJuSo wild type cells were incubated with increasing amounts of compound, stained with proteasome activity probe, lysed, resolved and measured as described. For the compounds on the left a concentration dependent increase in signal is observed. For the compounds on the right the signal remains relatively stable. The compounds proflavine and fenofibrate are not present in this Figure.

FIG. 17: Results from SDS-PAGE analysis of activation dynamics MelJuSo wild type cells were incubated with 5 μM Win 62,577 for 1 hour, followed by a one or two hour washout, staining with the proteasome activity probe and lysis of the cells. The strong activation seen after 1 hour incubation has disappeared after two hours of washout. NT=No activator, A=activator, no washout. WO 1 hr=1 hour washout and WO 2 hrs=2 hours washout.

FIG. 18: AMC conversion by cell lysates. 5 μM of compound was added to MelJuSo cell lysate, incubated for 45 minutes at 37° C. after which fluorogenic substrates were added. The conversion was measured for 90 minutes and results normalized to untreated controls. Experiment was performed in quadruplicate and error bars represent SD.

FIG. 19: (A) Cell viability of MelJuSo wild type cells incubated with 1 μM MG132 and 5 μM of the different proteasome activators. The presence of the latter results in an increase in resazurin conversion. (B) MelJuSo wild type cells incubated with increasing concentration of MG132. Results were plotted as percentage compared to a DMSO control.

FIG. 20: mRNA levels for proteasome β5 subunits in Hela cells after 16 hours exposure to 1 μM activator. mRNA levels were quantified using β-glucuronidase (GUS) as reference gene and depicted as (PSMB5/GUS)*100. Error bars represent SD of three replicates. N=1

### DETAILED DESCRIPTION

One aspect of the invention is directed to a composition for increasing the activity of 20S and/or 26S proteasome above basal levels which comprises of a compound identified as being activators of the 20S and/or 26S proteasome.

The compounds used in the compositions of the invention include salt forms of the compound, a specific stereoisomer of the compound, analogs, derivatives and prodrugs thereof. Examples of these forms of the compounds include, but are not limited to compounds where the functional group of the compounds has been protected—see e.g. Protective Groups in Organic Synthesis (Fourth Edition), Theodora W. Greene and Peter G. M. Wuts, Wiley-Interscience (October 2006).

Other examples of analog, derivative and prodrug forms of the compound, include, but are not limited to glycosylated forms of the compound. In another embodiment of this aspect of the invention, the glycosylated forms are those forms which serve to enhance the water-solubility of the compound.

Compounds suitable for increasing the activity of 20S and/or 26S proteasome, include, but are not limited to calcium channel modulators, cAMP inhibitors, antiandrogens (compounds that block the synthesis or action of androgens), p38 kinase inhibitors, methylbenzonium, proflavine, and PD 98059 (structures of latter three compounds shown below).

| | |
|---|---|
| Methylbenzethonium | 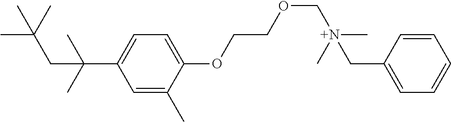 [SMILES: Cc1cc(C(C)(C)CC(C)(C)C)ccc1OCCOC[N+](C)(C)Cc1ccccc1] |
| Proflavine | 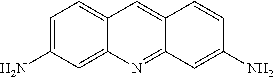 [SMILES: Nc1ccc2cc3ccc(N)cc3nc2c1] |
| PD98059 | 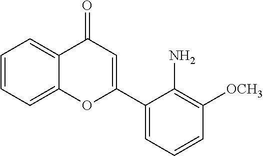 [SMILES: COc1cccc(-c2cc(=O)c3ccccc3o2)c1N] |

In one embodiment of the invention, the p38 kinase inhibitors have the general formula (I):

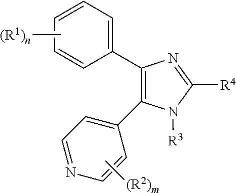

wherein,
R¹ is halogen, hydroxyl, amino, C1-C4 alkyl;
R² is halogen, hydroxyl, amino, C1-C4 alkyl;
R³ is hydrogen or C1-C4 alkyl;
R⁴ is phenyl or phenyl substituted with halogen, hydroxyl, amino, C1-C4 alkyl, nitro, sulfinyl, C1-C4 alkylsulfinyl; sulfonyl or C₁-C₄ alkylsulfonyl; or
R³ and R⁴ together form a 5-6 membered ring with at least one additional heteroatom selected from the group consisting of O, N and S;
n is 0-5; and
is 0-4.

In another embodiment of the p38 kinase inhibitors of general formula (I):
R¹ is halogen;
   R² is halogen;
   R³ is hydrogen or CH₃;
   R⁴ is phenyl substituted with halogen, hydroxyl, amino, C₁-C₄ alkyl, nitro, methylsulfinyl or methylsulfonyl; or
   R³ and R⁴ together form a 5-6 membered ring with at least one additional heteroatom selected from the group consisting of O, N and S;
   n is 0-2; and
   m is 0-1.

In another embodiment of the p38 kinase inhibitors of general formula (I):

### R¹ is F;

R³ is hydrogen or CH₃;
R⁴ is phenyl substituted with F, hydroxyl, amino, CH₃, nitro, or methylsulfinyl; or
R³ and R⁴ together form a 5 membered ring with S as one additional heteroatom;
n is 0-1; and
m is 0.

In another embodiment of the p38 kinase inhibitors are selected from the group consisting of:

| | |
|---|---|
| PD169316 | 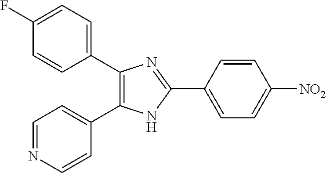 [SMILES: O=[N+]([O-])c1ccc(-c2nc(-c3ccc(F)cc3)c(-c3ccncc3)[nH]2)cc1] |
| SB203580 | 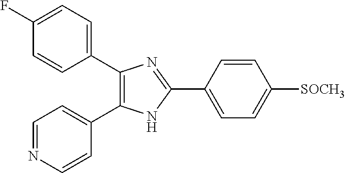 |
| SB202190 | 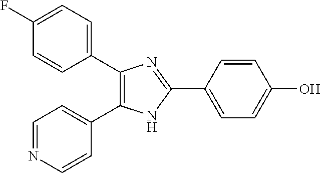 [SMILES: Oc1ccc(-c2nc(-c3ccc(F)cc3)c(-c3ccncc3)[nH]2)cc1] |
| SKF86002 | 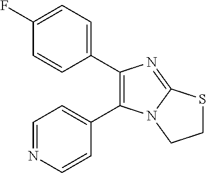 [SMILES: Fc1ccc(-c2nc3n(c2-c2ccncc2)CCS3)cc1] |

In one embodiment of the invention, the calcium channel modulators are selected from the group consisting of cyclosporin A, loperamide, metergoline, pimozide, Win 62,577 and verapamil (structures shown below).

| | |
|---|---|
| Cyclosporin A | 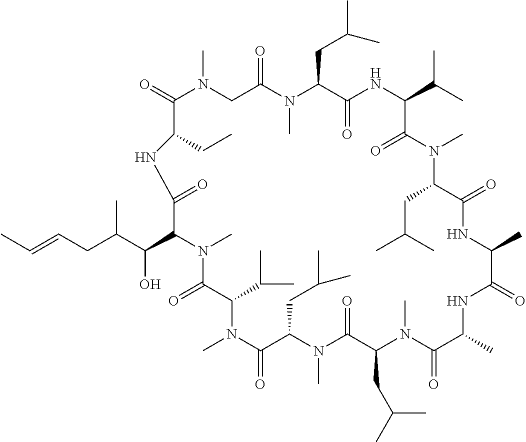 [SMILES: C/C=C/CC(C)[C@H](O)C1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] |
| Loperamide | 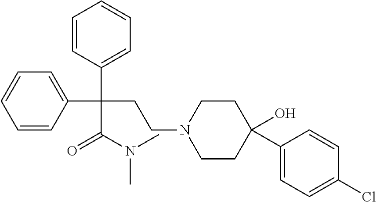 [SMILES: CN(C)C(=O)C(CCN1CCC(O)(c2ccc(Cl)cc2)CC1)(c1ccccc1)c1ccccc1] |
| Metergoline | 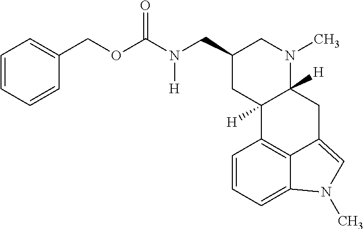 [SMILES: CN1C[C@H](CNC(=O)OCc2ccccc2)C[C@@H]2c3cccc4c3c(cn4C)C[C@H]21] |
| Pimozide | 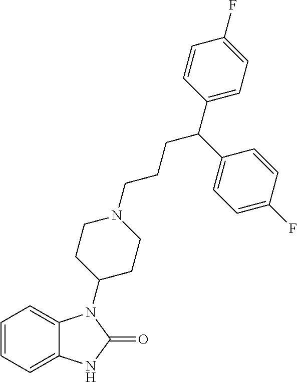 [SMILES: O=c1[nH]c2ccccc2n1C1CCN(CCCC(c2ccc(F)cc2)c2ccc(F)cc2)CC1] |
| Win 62,577 | 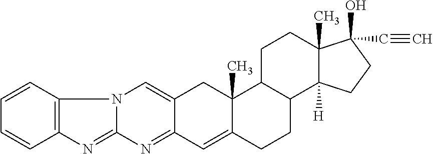 [SMILES: C#C[C@]1(O)CC[C@H]2C3CCC4=Cc5nc6nc7ccccc7n6cc5C[C@]4(C)C3CC[C@@]21C] |
| Verapamil | 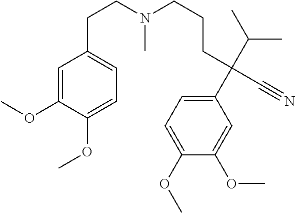 [SMILES: COc1ccc(CCN(C)CCCC(C#N)(c2ccc(OC)c(OC)c2)C(C)C)cc1OC] |

In one embodiment of the invention, the cAMP inhibitors are selected from the group consisting of cyclosporin A, cyproterone, dipyrimadole, DPCPX, fenofibrate, medroxyprogesterone, mifepristone and pimozide (structures not shown above are shown below).

| | |
|---|---|
| Cyproterone | 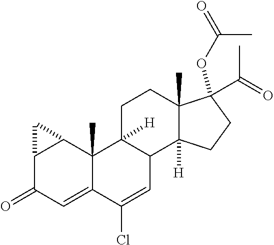 |
| Dipyrimadole | 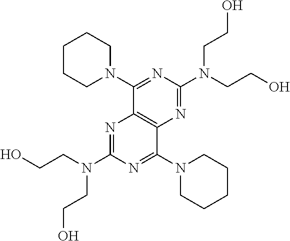 [SMILES: OCCN(CCO)c1nc(N2CCCCC2)c2nc(N(CCO)CCO)nc(N3CCCCC3)c2n1] |
| DPCPX | 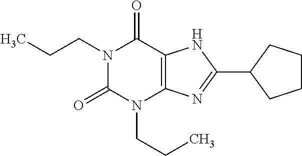 [SMILES: CCCn1c(=O)c2[nH]c(C3CCCC3)nc2n(CCC)c1=O] |
| Fenofibrate | 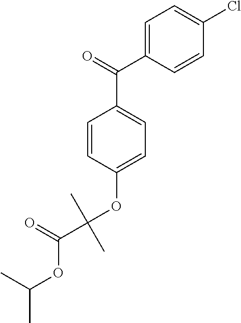 [SMILES: CC(C)OC(=O)C(C)(C)Oc1ccc(C(=O)c2ccc(Cl)cc2)cc1] |
| Medroxyprogesterone | 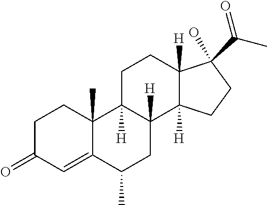 |
| Mifepristone | 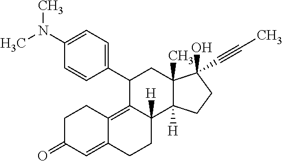 |

In one embodiment of the invention, the antiandrogens are selected from the group consisting of cyproterone, mifepristone and medroxyprogesterone.

In another embodiment of the invention, the composition comprises of a compound which targets at least one deubiquitinating enzyme (DUB).

In another embodiment of the invention, the composition comprises of a compound which targets more than one deubiquitinating enzyme (DUB).

With regard to the compositions of the invention, these compositions may be prepared according to any method known in the art for the manufacture of pharmaceutical compositions. Remington—The Science and Practice of Pharmacy (21^{st} Edition) (2005), Goodman & Gilman's The Pharmacological Basis of Therapeutics (11^{th} Edition) (2005), Ansel's Pharmaceutical Dosage Forms and Drug Delivery Systems (9^{th} Edition), edited by Allen et al., Lippincott Williams & Wilkins, (2011), Solid-State Chemistry of Drugs (2nd Edition)(1999), each of which is hereby incorporated by reference.

Another aspect of the invention is directed to a method for increasing the activity of the 20S and/or 26S proteasome above basal levels by administering a therapeutically effective amount of the above described composition for increasing the activity of 20S and/or 26S proteasome to a patient in need thereof.

In one embodiment of the invention, the increased activity occurs within a time range selected from the groups consisting of two hours of administration, one hour of administration and 30 minutes of administration.

In another embodiment of the invention, the activation of 20S or 26S proteasome is reversible.

In another embodiment of the invention, the concentration of the activator compound in the composition is selected from the ranges consisting of from about 0.01 to about 20.0 μM; from about 0.05 to about 10.0 μM; and from about 0.1 to about 5.0 μM.

In another embodiment of the invention, the composition used in the method comprises of a compound which targets at least one deubiquitinating enzyme (DUB).

In another embodiment of the invention, the composition used in the method comprises of a compound which targets more than one deubiquitinating enzyme (DUB).

In another embodiment of the invention, the activator compound increases the proteasome activity between two and five fold relative to no treatment.

Another aspect of the invention is directed toward a method of modulating the 20S and/or 26S proteasome by administering a therapeutically effect amount of the 20S and/or 26S proteasome activator compound in order to modulate the 20S and/or 26S proteasome as necessary to a patient in need thereof.

In this aspect of the invention, in addition to the continuous activation of the 20S and/or 26S proteasome, the activation of the 20S and/or 26S proteasome can be modulated by administering a composition for a period of time to reach the desired level of activity followed by a period of time with a cessation of administration of the composition. The administration and cessation of administration constitutes one cycle of treatment and one or more cycles of treatment may be administered to the patient as required.

Another aspect of the invention is directed toward a method of direct activation of the 20S and/or 26S proteasome by administering a therapeutically effective amount of 20S and/or 26S proteasome activator compound in order increase the activity of the 20S and/or 26S proteasome above basal levels as necessary to a patient in need thereof.

Another aspect of the invention is directed toward a method of indirect activation of the 26S proteasome by administering a therapeutically effect amount of 26S proteasome activator compound in order to increase the activity of the 26S proteasome above basal levels as necessary to a patient in need thereof.

While not wishing to be bound by theory, the increase of activity of 20S and/or 26S proteasome by the compounds/compositions of the invention can occur by one or more pathways which include, but are not limited to local increase of the Ca²⁺ concentration in cells, increasing the assembly of 26S proteasome from 19S RP and 20S CP units, direct or indirect phosphorylation of 19S RP and 20S CP units, inhibiting endogenous proteasome inhibitors or interfering with the activity of deubiquinating enzymes (DUBs).

As proper disposal of “broken” and “undesired” proteins is critical to life, the proteasome plays an important role in many essential cellular processes such as the cell cycle [3, 33, 40], misfolded protein response [3, 39], apoptosis [3, 41-43], differentiation [3, 6], development [3, 6, 44], response to stress [36, 45-47], regulation of different stages of gene expression [3, 45] and in the immune response [5, 36, 48]. Because of the important role of the proteasome in the cell it is often involved in disease. Changes in the UPS can lead to the development of inflammatory and autoimmune diseases [48] and are involved in cancer [41]. As the proteasome is involved in the regulation of many proteins that play a role in cancer such as p53, p27^{kip1}, pVHL and BRCA1/BARD1 [6], the disruption of normal proteasome function can contribute to the malignant transformation of cells [3, 49].

The 26S proteasome has also been implicated to play an important role in various neurodegenerative disorders [50]. A common feature of these disorders is the formation of large intracellular protein aggregates containing both ubiquitin and proteasome. Based on this observation, it is hypothesized that the impairment of the UPS plays a role in the development for some types of inheritable Parkinson's and Alzheimer disease [6].

Therefore, another aspect of the invention is the treatment of neurodegenerative diseases/disorders and diseases characterized by protein aggregation and/or protein deposition, autoimmune diseases, infection diseases, cancer and inflammation by the activation of the 20S and/or 26S proteasome which comprises of administering a therapeutically effective amount of a compound which is an activator of 20S and/or 26S proteasome to a patient in need thereof.

In one embodiment of this aspect of the invention, the treatment of neurodegenerative diseases and diseases characterized by protein aggregation and/or protein deposition can include, but is not limited to Alzheimer's disease, Parkinson's disease, amyotrophic lateral sclerosis (ALS), Huntington's disease, transmissible spongiform encephalopaties (TSEs), Creutzfeld-Jakob disease, systemic amyloidosis, prion based diseases and diseases caused by polyglutamine repeats.

In one embodiment of this aspect of the invention, autoimmune diseases include, but are not limited to those autoimmune diseases selected from the group consisting of those diseases, illnesses, or conditions engendered when the host's systems are attacked by the host's own immune system which comprises of, but is not limited to alopecia areata, ankylosing spondylitis, arthritis, antiphospholipid syndrome, autoimmune Addison's disease, autoimmune hemolytic anemia, autoimmune inner ear disease (also known as Meniers disease), autoimmune lymphoproliferative syndrome (ALPS), autoimmune thrombocytopenic purpura, autoimmune hemolytic anemia, autoimmune hepatitis, Bechet's disease, Crohn's disease, diabetes mellitus type 1, glomerulonephritis, Graves' disease, Guillain-Barre syndrome, inflammatory bowel disease, lupus nephritis, multiple sclerosis, myasthenis gravis, pemphigus, pemicous anemia, polyarteritis nodosa, polymyositis, primary billiary cirrhosis, psoriasis, Raynaud's Phenomenon, rheumatic fever, rheumatoid arthritis, scleroderma, Sjogren's syndrome, systemic lupus erythematosus (SLE), ulcerative colitis, vitiligo, and Wegener's granulamatosis.

In one embodiment of this aspect of the invention, the infectious disease includes, but is not limited to those infectious diseases selected from the group consisting of those diseases associated with defective antigen presentation via MHC molecules.

In one embodiment of this aspect of the invention, the treatment of cancer can include, but is not limited to leukemia, carcinoma (including that of bladder, breast, colon, kidney, liver, lung, ovary, pancreas, stomach, cervix, thyroid, prostate, head, neck and skin); hematopoietic tumors of lymphoid lineage (including acute lyphocytic leukemia), B-cell lymphoma, and Burkett's lymphoma, hematopoietic tumors of myeloid lineage, including acute and chronic myelogenous leukemias and promyelocytic leukemia; tumors of mesenchymal origin (including fibrosarcoma and rhabdomyasarcoma); and other tumors (including melanoma, seminoma, teratocarcinoma, osteosarcoma, neuroblastoma and glioma).

In one embodiment of this aspect of the invention, the treatment of inflammation can include, but is not limited to rheumatoid arthritis, spondyloathopathies, gouty arthritis, osteoarthritis, systemic lupus erythematosis, and juvenile arthritis, bronchitis, bursitis, gastritis, inflammatory bowel disease, ulcerative colitis, acne vulgaris, asthma, autoimmune dieases, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

All of the embodiments described above for the compositions of the invention may be used in conjunction with the embodiments described above for the treatment of neurodegenerative diseases/disorders and diseases characterized by protein aggregation and/or protein deposition, autoimmune diseases, infection diseases, cancer and inflammation.

Another aspect of the invention is a method for identifying compounds that increase the activity of the 20S and/or 26S proteasome above basal levels.

Besides fluorogenic substrates, two other methods used to monitor proteasome activity are small molecule probes and models based on recombinant reporter proteins [14]. The latter remains confined to genetically altered cells or organisms. Therefore results obtained in this type of experiment do not necessarily represent the situation in vivo. Also, these methods do not allow for the profiling of patient material. [14].

One example of a small molecule probe is the Me₄BodipyFL-Ahx₃Leu₃VS proteasome activity probe [14]. This fluorescent probe is cell membrane permeable and binds irreversibly to all three catalytically active subunits of proteasomes in living cells. It can be used to measure proteasome activity in both live cells and cell lysates. Furthermore, this proteasome activity probe is compatible with a variety of techniques including gel-based assays, confocal laser scanning microscopy and flow cytometry [14]. In contrast to fluorogenic substrates and recombinant reporter proteins, small molecule probes can be used in live cells. In addition, these probes allow for patient material profiling. The main advantage of the proteasome activity probe is that it provides a robust and sensitive way to monitor proteasome activity in live cells. This allows for the high-throughput screening (HTS) of large compound and small interfering RNA (siRNA) libraries. Such screens will help with identifying new 20S and/or 26S proteasome activators as well as provide insight in the regulation of the proteasome in general.

In one embodiment of this invention, the method of identification comprises:
i. obtaining a compound (a) from a compound library or other source;
ii. obtaining a cell type which expresses constitutive proteasome and incubating the cell type with the compound (a);
iii. addition of a fluorescent probe to a cell culture of the cell-type, which binds covalently to the catalytic subunits of the 20S CP of the 26S proteasome to transform the catalytic subunits of the 20S or 26S proteasome into a 20S or 26S proteasome-fluorescent probe complex;
iv. measuring fluorescence (FL-1) of the 20S or 26S proteasome-fluorescent probe complex by flow cytometry and/or confocal microscopy and image analysis of a combination thereof and measuring the forward scatter, side scatter to create a score for proteasome activity and converting the score to an FL-1 log 2 ratio relative to the average of untreated cells. This score is converted to an FL-1 log 2 ratio relative to the average of untreated cells;
v. identifying compound (a) which have a FL-1 log 2 ratio greater than 1.00;
   A FL-1 log 2 ratio corresponds to a 2-times increased proteasomal activity compared to proteasomal activity that is measured when no compound is added and is hereby chosen as a threshold;
vi. validating the identified compound (a) in step v. by:
   a. repeating steps i.-iv.; and
   b. repeating steps i.-iii., followed by lysing the cells to form a cell lysate which is resolved by SDS-PAGE and subsequently analyzed by fluorescent scanning of the resulting gel;
vii. identifying compound (a) which still have a FL-1 log 2 ratio greater than 1.00 after step vi. a. and have bands that show dose-dependent increased fluorescence for the β2 and β5 subunits of the 20S or 26S proteasome after step vi. b.
   (Compounds with a FL-1 log 2 ratio greater than 1.00 increase the fluorescence four times or more compared to untreated cells.)

In another embodiment of this invention, step iii. reports proteasome activity beyond basal levels.

In another embodiment of this invention, the binding of the fluorescent probe is irreversible.

In another embodiment of this invention, the fluorescent probe is a vinylsulfone (VS) based probe, which includes, but is not limited to Me₄BodipyFL-Ahx₃Leu₃VS.

In another embodiment of the invention, the concentration of compound (a) relative to the cell type is selected from the ranges consisting of about 0.01 to about 20 μM, about 0.05 to about 10 μM; and about 0.1 to about 5.0 μM.

In another embodiment of the invention, the lysing is achieved by sonication or any other lysis method.

The invention is further described by the following non-limiting examples which further illustrate the invention, and are not intended, nor should they be interpreted to, limit the scope of the invention.

### Experimental Procedures

Described below are the procedures for establishing increased proteasome activity.

For the activation of 20S and/or 26S proteasome, two chemical compound libraries (LOPAC and JHCCL) were screened for the presence of compounds which activate the 20S and/or 26S proteasome in cells. For this screening a FACS based activity assay was used using the fluorescent proteasome activity probe Me₄BodipyFL-Ahx₃Leu₃VS as readout. Hits from both libraries were validated using FACS, fluorogenic substrates and SDS-PAGE based experiments. By means of FACS and SDS-PAGE it was investigated if the observed activation was concentration dependent and using fluorogenic substrates it was assessed if the compounds were able to directly activate the proteasome. In an attempt to investigate the pathways underlying proteasome dynamics, FACS analysis was used to monitor the increase of activation over time. To further test the dynamics of increased proteasome activity, a washout experiment was performed. Furthermore, the effect of proteasome activators on cell viability of cells exposed to proteasome inhibitors was investigated using a Cell-Titer-Blue cell viability assay.

### Material and Methods

### Workflow Compound Screens

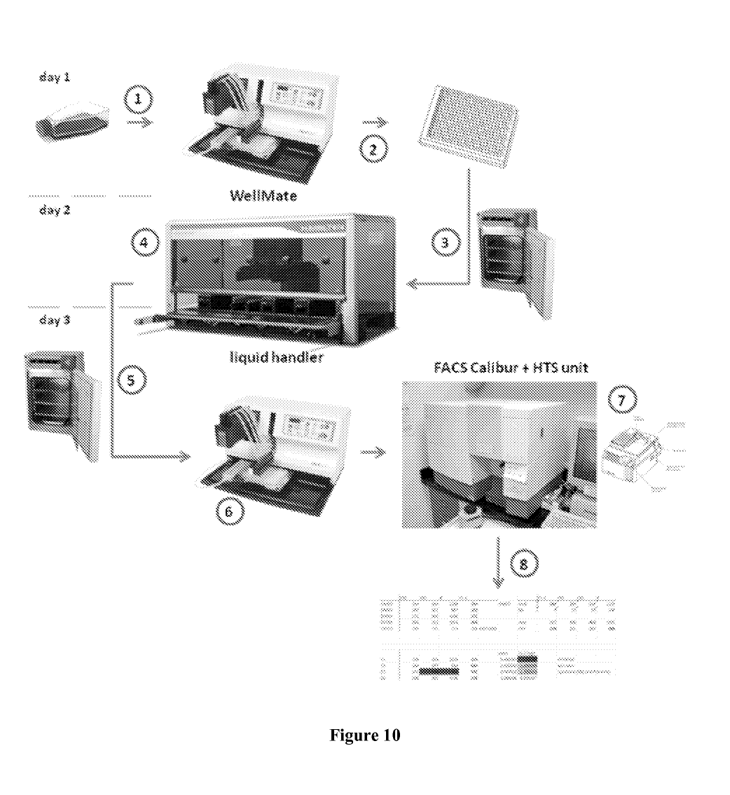

For the HTS screening a three day protocol was used. A schematic representation of the workflow used to screen two compound libraries is given in FIG. 10.

### Day 1: Seeding Cells in 384 Wells Plate

MelJuSo cells are washed, trypsinized and resuspended in medium containing FBS and antibiotics. After counting the concentration is adjusted to 200,000 cells per mL. Using the Wellmate microplate dispenser fitted with small bore nozzle tubing, 50 μL of cell suspension is transferred to each well of a black 384 wells plate (10,000 cells/well). Cells are left in the incubator for approximately 24 hours.

### Day 2: Adding Compounds to Cells

Plates containing compounds are removed from the freezer approximately 24 hours prior to exposure. A brief spin down will prevent any liquid from being lost when the plate cover is removed. Using a Hamilton liquid handling workstation the compounds are diluted to appropriate concentrations. To “wash” the cells prior to exposure 30 μL of medium is removed from each well and is replaced with fresh medium. Then the compounds are added to the medium of the cells (final concentration 5 μM). As a negative control, cells were incubated with 750 nM of MG132. Cells are left in the incubator for approximately 16 hours.

### Day 3: Staining Cells, Harvesting Cells and FACS Measurements

To stain the cells, Me₄BodipyFL-Ahx₃Leu₃VS activity probe is dissolved in medium at a concentration of 1200 nM. Using the Wellmate microplate dispenser fitted with small bore nozzle tubing, 10 μA of probe suspension is transferred to each well resulting in a final probe concentration of 200 nM. Plates are left to incubate for two hours. Then the medium is removed and the cells are washed one time with PBS. The PBS is removed and 10 μL of trypsin is added to each well. The plates are left on a shaker for 2 minutes, placed in an incubator for 5 minutes and put on a shaker again for 2 minutes. 40 μL of PBS containing 2% FBS is added per well and the plates are put on the shaker for 2 minutes. Finally, 20 μL of PBS containing 4% formaldehyde is added and the plates are left to shake for at least 15 minutes. Plates are left on a shaker at 4° C. until they can be measured.

The FITS unit is installed on the FACS and everything is turned on. To remove air from the system and ascertain that everything works fine the system is primed twice. One by one the plates are brought down and measured. In between plates a “daily clean” cycle is performed to prevent the system from becoming clogged. After the last plate the system should be cleaned again.

### Analysis

Analysis is always performed per plate. Throughout the analysis the following parameters are considered:
Forward Scatter (FSC): size of the cell
Side Scatter (SSC): granularity of the cell
Number of events (#)
Probe signal (FL-1): proteasome activity
FL-2 and FL-3: signal in other channels

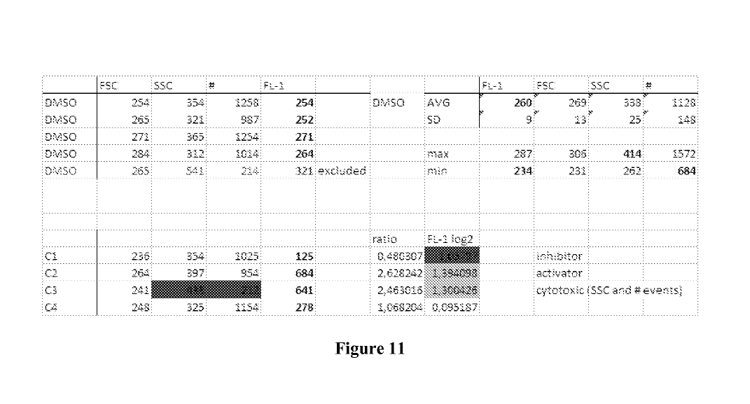

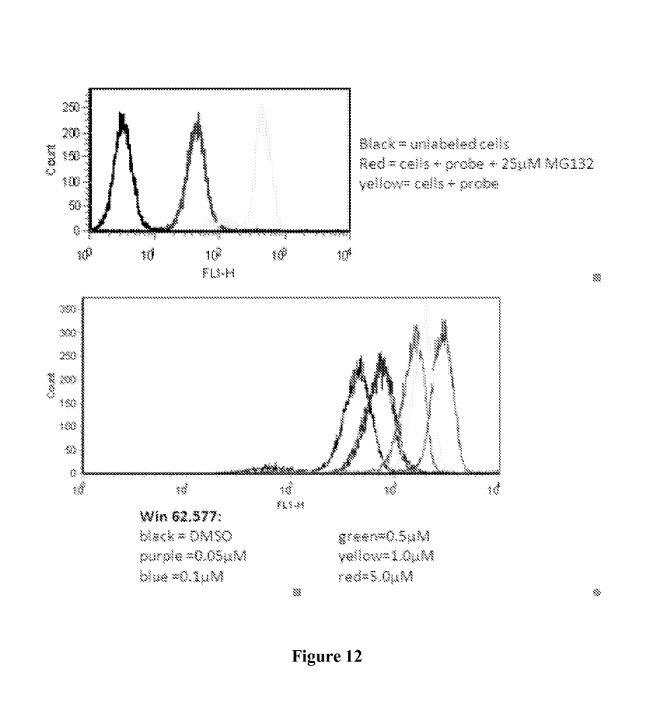

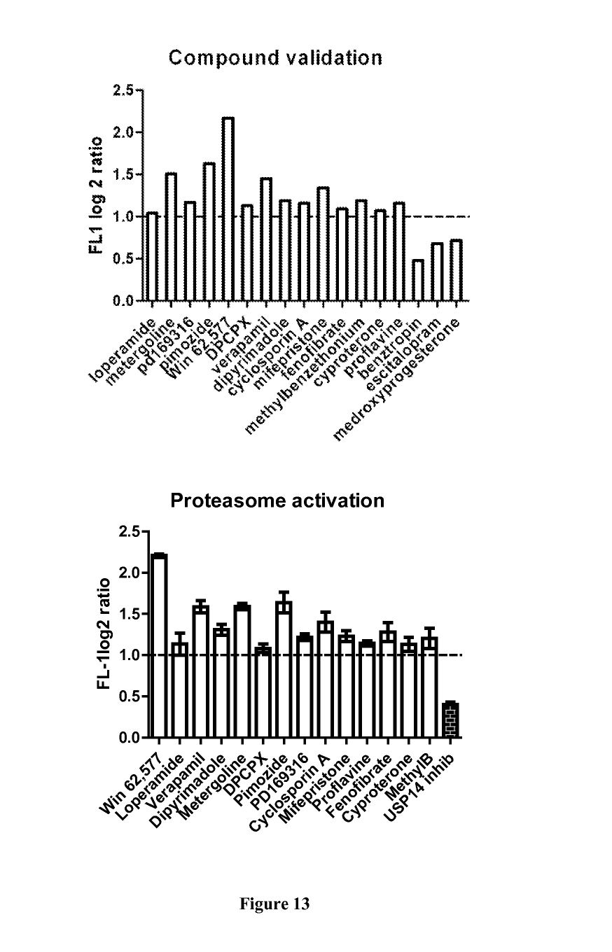

All DMSO samples are checked and aberrant wells (low # events, high SSC, no signal) are excluded. The average of the FL-1 from all remaining DMSO samples is used to calculate the “ratio vs. DMSO” and the FL-1 log 2 ratio of the experimental samples. This will result in DMSO samples having a FL-1 log 2 value around zero. Compounds are considered “activators” or “hits” if the FL-1 log 2 ratio is >1, and inhibitors if the FL-1 log 2 ratio is >−1. This cutoff is strict enough to ensure that all remaining hits should be strong activators. An example of how the analysis was performed is given in FIG. 11.

### Exclusion Criteria:

Cytotoxicity (based upon SSC and # of events)
   Exclude sample if SSC>average of DMSO+3× Standard Deviation (SD) and/or if # events <DMSO−3×SD
Autofluorescence (very high FL-1, elevated FL-2 and FL-3)
   Exclude sample if FL-2 and/or FL-3> average of DMSO+3×SD
Mis-hit (no duplo present or only one sample elevated)
   All compounds with a FL-1 log 2 ratio higher than 1 and who pass all of the exclusion criteria are considered “activators” or “hits”. While this cutoff is arbitrary it will insure that the compounds that are identified as hits have a strong effect on 26S proteasome activity.

### Cell Culture

MelJuSo Wild Type (human, multiple myeloma) and MelJuSo β1gfp cells were maintained in Dulbecco's Modified Eagle Medium (DMEM, Gibco) supplemented with 10% FBS and 100 μg/ml penicillin/streptomycin and were kept at 5% CO₂ and 37° C.

### Proteasome Labeling in Live Cells

Cells were washed once with phosphate buffered saline (PBS, 1 GIBCO tablet in 500 mL MilliQ). Fresh medium containing the compound at the indicated concentration was added. After the incubation time 200 nM of Me₄BodipyFL-Ahx₃Leu₃VS activity probe was added to the cells and after two hours of incubation cells were harvested for subsequent analysis by FACS or post-lysis fluorescence detection.

### Harvesting and Lysing of MelJuSo Cells

Cells were washed once with PBS, trypsin (6 well plate: 500 μL, 12 well plate: 200 μL, 24 well plate: 100 μL) was added and the plate was placed at 37° C. for 5 minutes to allow the cells to detach. When fully detached the cells were resuspended in fresh culture medium. Medium with cells was collected and after a centrifugation step (1100 ref for 2 minutes) the cells (pellet) were resuspended in cold HR buffer (50 mM Tris pH=7.4, 5 mM MgCl₂, 250 mM sucrose, 1 mM DTT and 2 mM ATP). Lysis of the cells is achieved by sonification (Bioruptor, high intensity for 5 minutes with an ON/OFF cycle of 30 seconds) at 4° C. After a centrifugation step (13.600 rpm for 10 minutes) to remove cell debris the protein concentration (absorbance at 280 nm) of the supernatant is measured with a NanoDrop spectrophotometer. Fresh HR buffer without any cells was used as a blank,

### Monitoring Proteasomal Activity in Cell Lysates Using Fluorogenic Substrates

Chymotrypsin-like (β5), trypsin-like (β2), and caspase-like (β1) proteolytic activities of the proteasome were measured in freshly prepared cell lysates as described above. Fluorogenic peptide substrates LLVY-AMC (100 μM), VGR-AMC (100 μM) and LLE-AMC (50 μM) were used to measure the chymotrypsin-like, trypsin-like, and caspase-like activity, respectively. All the substrates were dissolved in Tris/MgCl₂ buffer. The substrates were added to the samples after the 45 minutes incubation step (40 μL of substrate solution containing 20 μg of protein, 40 uL buffer containing inhibitors, activators etc, 20 μl of buffer containing substrates, total volume 100 μL).

The release of AMC was monitored online over a 90 minute time period at 37° C. with measurements taken every 5 minutes. Fluorescence was measured using a Victor 1420 Multilabel Counter (Perkin Elmer) using excitation and emission wavelengths of 360 and 465 nm, respectively. Proteolytic activity was calculated from the slopes of the linear part of the curves. All results were expressed as percentage relative to untreated MelJusocells (100%). Non-specific activities were determined using 1 μM epoxomicin, which is considered to specifically inhibit all proteasomal activity at this concentration, and the background signal obtained was subtracted from each measurement. Data were analyzed by using GraphPad Prism software (GraphPad, La Jolla, Calif., USA).

### In Gel Fluorescence Measurements

Equal amounts of protein (15-20 μg) were denatured by heating the samples for 10 minutes at 71° C. in loading buffer (450 μL 4× loading buffer (Invitrogen), 45 μL β-Mercaptoethanol and 105 μl, MilliQ, 2 parts sample to 1 part loading buffer). The samples were loaded onto a 12% SDS-PAGE gel using the NuPAGE system from Invitrogen. MOPS (3-(N-morpholino)propanesulfonic acid) was used as a running buffer. Gels run at 180 V for approximately 1.5-2.0 hours and were directly imaged using the ProXPRESS 2D Proteomic imaging system (Perkin Elmer). Resolution was set at 100 μm, excitation at 480/30 and emission at 530/30. To verify protein loading, gels were stained with Coomassie Brilliant Blue. Images were analyzed using Totallab analysis software (Nonlinear Dynamics, Newcastle upon Tyne, UK) to quantify the intensity of the bands detected.

### FACS Measurements

Celts were washed once with PBS, trypsin was added and the plate was placed at 37° C. for 5 minutes to allow the cells to detach. When detached the cells were resuspended in PBS supplemented with 2% FBS. Cells were fixed with formaldehyde (final concentration 1% in PBS). All samples were measured using a FACSCalibur flow cytometer (BD Biosciences). For MelJuSo cells the following settings were used: FSC 8.02 E-1, SSC 375 1× and FL-1 450. The complete workflow used for the compound screens is described above in more detail.

### Survival Assay

MelJuSo cells (50,000 cells/ml) were simultanously incubated with 5 μM Win 62,577 and 0, 10, 30, 100, 300, 1000 nM MG132 for 24, 48 or 72 hours. After incubation, the resazurin solution (12.5 mg/100 ml resazurin) was added to the cells. The increase in fluorescence (590 nm) was measured after 8 hours using an EnVsion multilabel reader (Perkin Elmer). All results were expressed as percentage relative to untreated cells (100%).

### Results

Search for Compounds that (In)directly Activate 26S Proteasome Activity in Cells

Compounds that increase proteasome activity are rare and not well studied. In this study the following approach was used in order to identify potential proteasome activators: (1) Screen for compounds that activate the 20S and/or 26S proteasome within two chemical compound libraries (LOPAC and JHCCL), (2) validation of the compounds identified and (3) elucidation of pathways involved in 20S and/or 26S proteasome regulation. These parts will now be discussed in this order.

Screening for Compounds that Increase 20S and/or 26S Proteasome Activity in Cells

To identify compounds with 26S proteasome activating properties two compound libraries were screened. The LOPAC (Library of Pharmacologically Active Compounds) is a collection of 1280 well documented molecules that span a broad range of cell signaling and neuroscience areas. The JHCCL (Johns Hopkins Clinical Compound Library) consist of 1,937 FDA-approved drugs and 750 drugs that were either approved for use outside the USA or undergoing phase 2 clinical trials. [74]

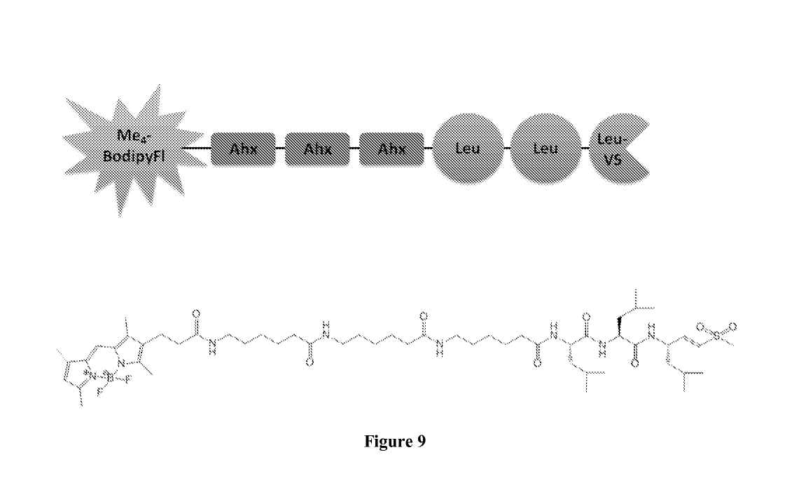

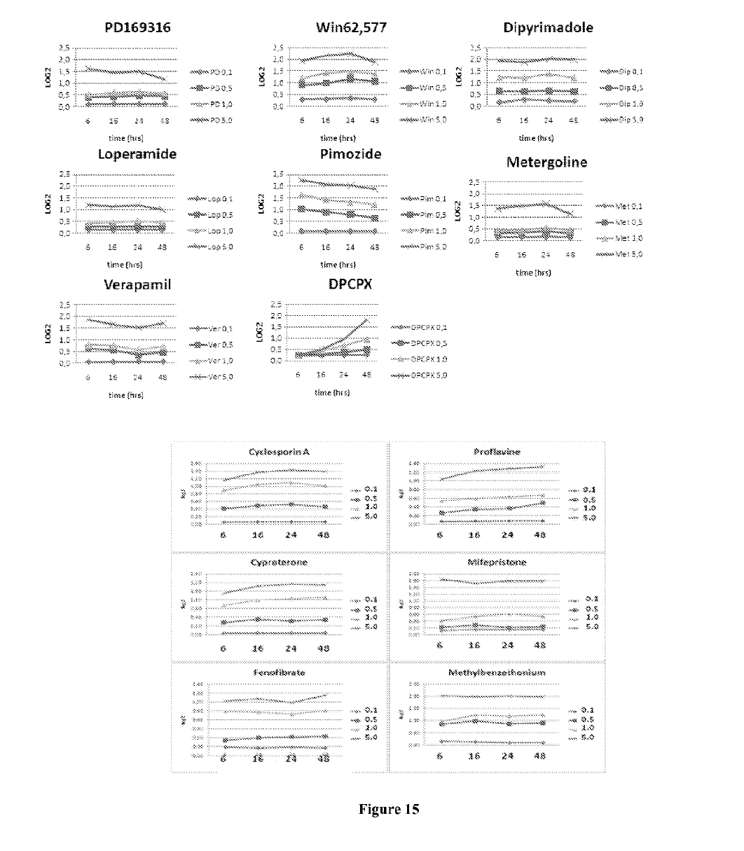

The compound libraries mentioned above are screened using a FACS-based activity assay. The proteasome activity probe Me₄BodipyFL-Ahx₃Leu₃VS activity probe is used to fluorescently label the proteasome. This probe binds irreversibly to the catalytic domains of the 20S CP. When proteasome activity is increased, increased probe binding results in an increase in fluorescent signal compared to untreated cells. When the proteasome is pre-treated with a proteasome inhibitor the probe is no longer able to bind to the catalytic subunits and a decrease in signal is observed. The structure of the proteasome activity probe is shown in FIG. 9 and representative examples of both an inhibition and an activation experiment is depicted in FIG. 15.

For the compound screens, MelJuSo wild type cells (expressing mainly constitutive proteasome) were incubated with 5 μM compound for 16 hours and subsequently stained with 200 nM proteasome activity probe for two hours. The fluorescence signal was then measured by FACS, along with several other parameters. The fluorescence score (FL-1) was first normalized to the average of untreated controls and subsequently converted in an FL-1 log 2 ratio. All compounds with an FL-1 log 2 ratio >1.00 and which did not meet any of the exclusion criteria (cytotoxicity, autofluorescence etc) were considered to be true “hits” and taken along for validation experiments. Flits typically increase the activity of the proteasome between two and five times compared to untreated controls.

From the LOPAC the following compounds were identified as hits: loperamide (opioid receptor ligand), metergoline (serotonin receptor antagonist), PD169316 (p36 MAP kinase inhibitor), pimozide (dopamine receptor antagonist), Win 62,577 (NK1 tachykinin receptor antagonist) and 8-cyclopenthyl-1,3-dipropylxanthine (adenosine receptor antagonist). From the JHCCL the following compounds were identified as hits: cyclosporin A (immunosuppressant), mifepristone (abortifacient, emergency contraceptive), fenofibrate (anti-cholestrol drug), methylbenzethonium (antiseptic), cyproterone (antiandrogen) and proflavine (antisceptic). The compounds verapamil (calcium channel modulator, Pgp inhibitor) and dipyrimadole (adenosine receptor inhibitor) were present in the two libraries and were identified as hits in both.

### Validation of Screening Hits

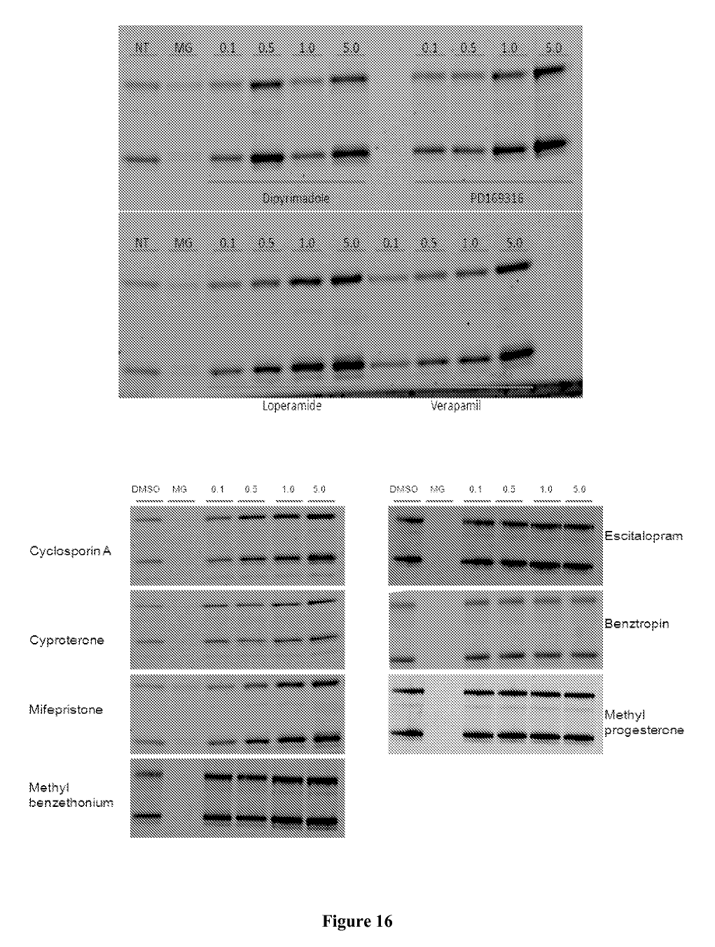

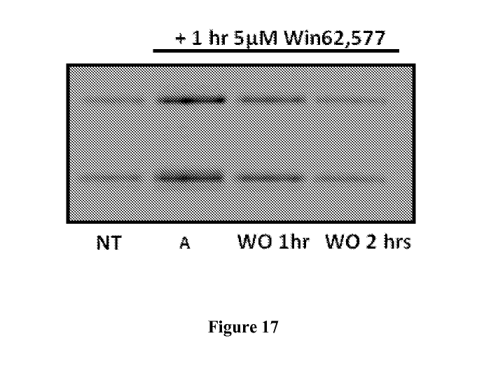

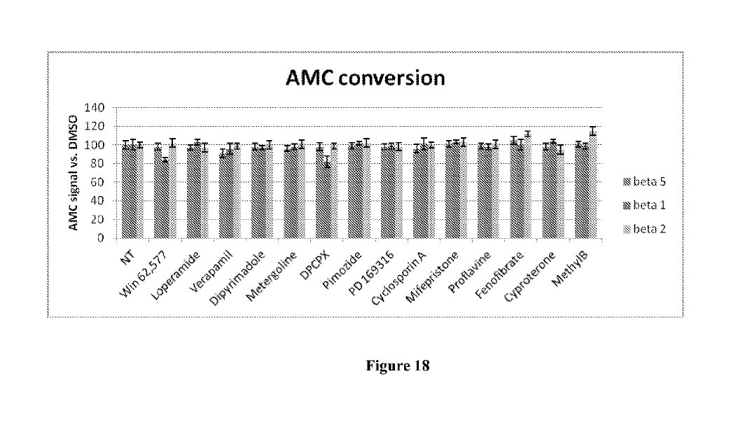

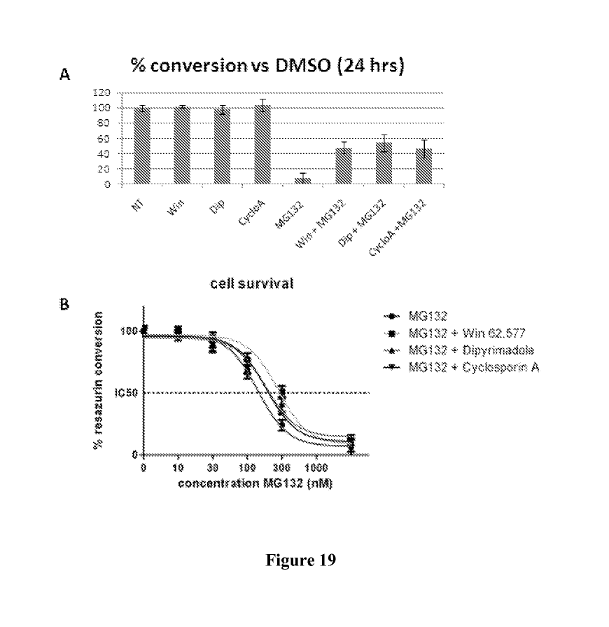

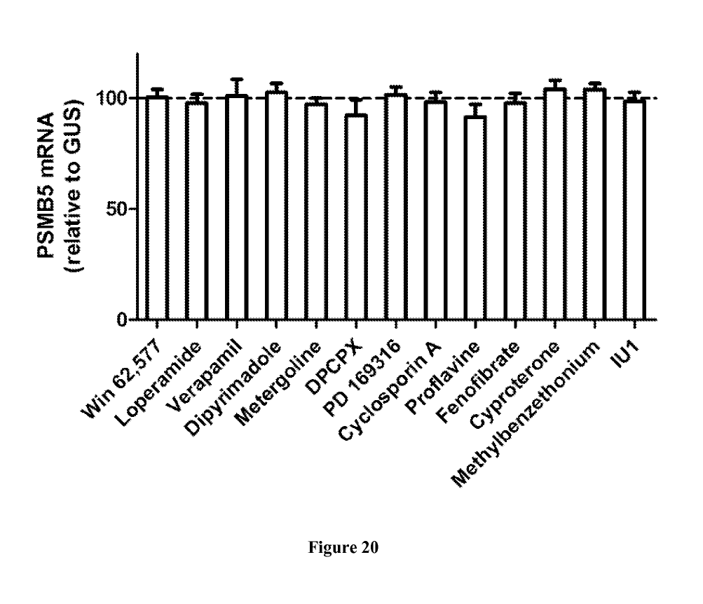

The hits from both libraries were first validated by FACS. The reason to verify the compounds by FACS was to check if the compound from the library and the freshly ordered compound gave a similar response. FIG. 16 shows the respective FL-1 log 2 ratios obtained after 16 hours of incubation with 5 μM compound for all hits. Most of the compounds showed similar FL-1 log 2 scores during the FACS validation experiment as during the screen. Additionally, FACS and SDS-PAGE validation experiments were done in which cells were incubated for 16 hours with 0.1, 0.5, 1.0 or 5.0 μM compound. These experiments would give us information about the dose-dependency of increased activity caused by the compounds.

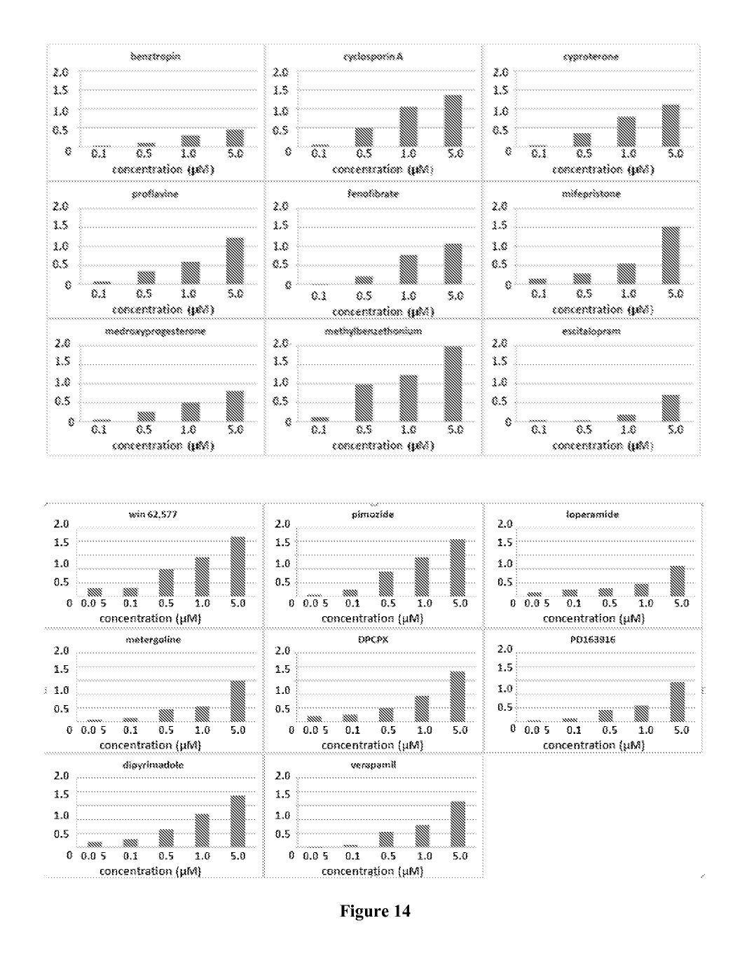

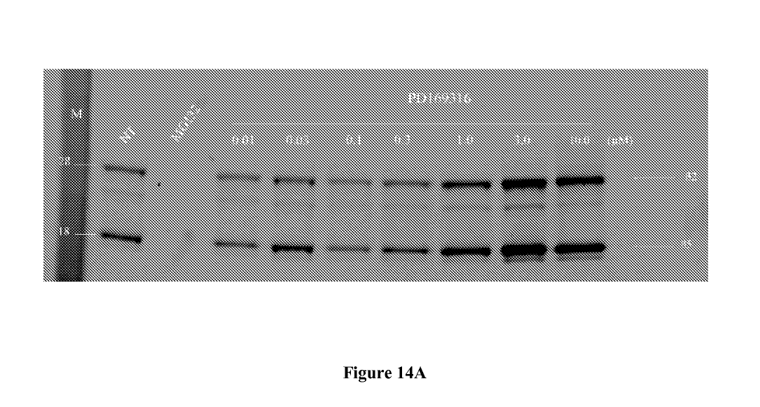

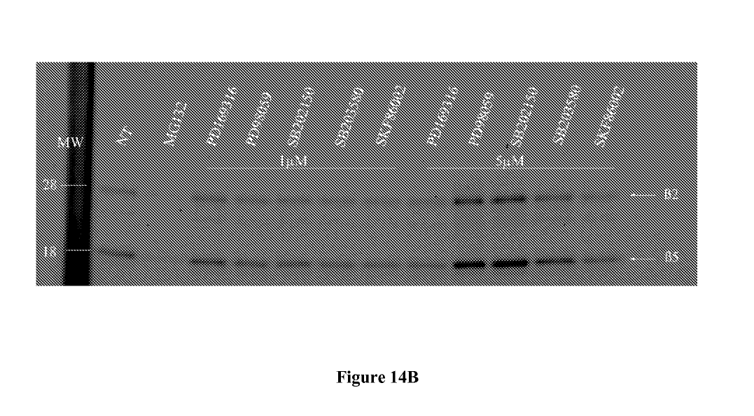

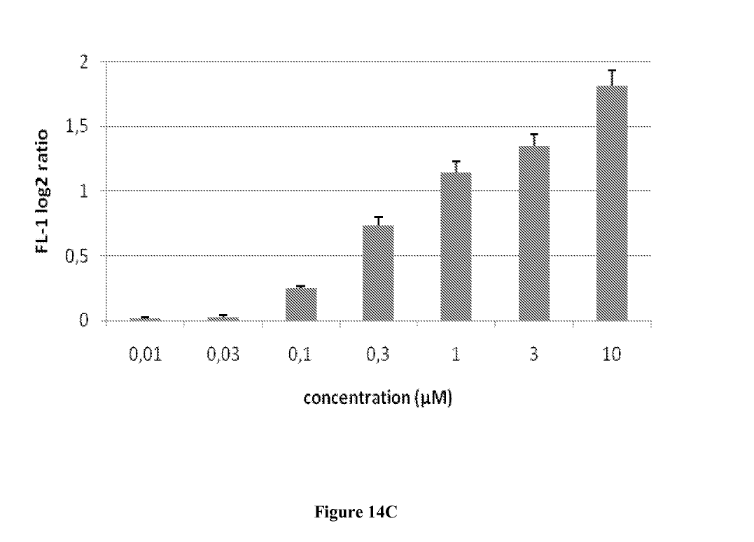

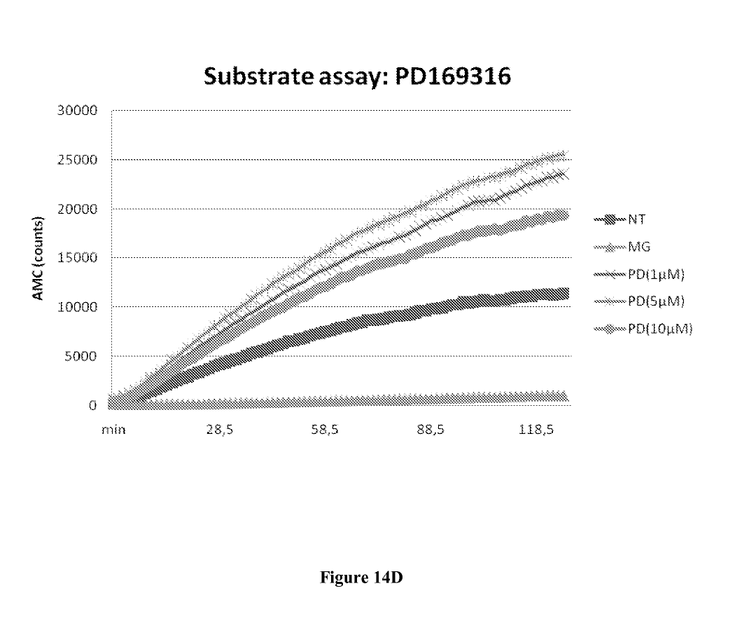

For three of the compounds the results are depicted in FIG. 17. Data for the other compounds can be found in the specification and in FIGS. 14 and 16. Most compounds showed a dose-dependent increase of the fluorescent signal on gel. However, the compounds benztropin, escitalopram and medroxyprogesterone that were initially identified in the JHCCL screen displayed weak(er) proteasome activation during validation. Even the highest concentration failed to result in a FL-1 log 2 ratio >1.0. These compounds were therefore not taken along for further experiments. The SDS-PAGE result also shows a dose-dependent increase of proteasome activity. Furthermore, the increase in activity is more or less equal for all catalytic subunits of the proteasome.

### Comparison of Screening Hits and USP14 Inhibitor

Recently, an USP14 inhibitor was reported to activate the proteasome in cells [56] and can therefore be used as a benchmark. The function of USP14 is to rescue substrates from 26S proteasome degradation by removing the poly-ubiquitin tail from tagged proteins. After validation, the 14 compounds identified in the screen were tested together with this USP14 inhibitor [21]. The results of this experiment are depicted in FIG. 18. When comparing the FL-1 log 2 scores, the USP14 inhibitor scores relatively low compared to the compounds identified in this study. This would indicate that the USP14 inhibitor activates the proteasome to a much lesser extent than the other compounds.

### Dynamics of the Compound PD169116 on Proteasome Regulation

To gain insights into the effects of the PD169316 on distinct proteasomal subunits, an SDS-PAGE-based profiling experiment was done. Human melanoma (MEL-JUSO) cells were incubated for 16 h with increasing PD169316 concentrations of 0.01, 0.01, 0.1, 0.1, 1.0, 3.0, and 10.0 μM; untreated cells and cells incubated with the proteasome inhibitor MG132 (1 μM) served as a positive and negative control, respectively. The proteasomal subunits are then labeled using the fluorescent Me_{q}-Bodipy-FL-Ahx₁-Leu₁-VS probe, which selectively binds only to active β-subunits of the proteasome [84]. The fluorogenic-labeled proteasome subunits were visualized directly in-gel by fluorescence scanning. Incubation with PD169316 concentrations of 1.0, 1.0 and 10.0 μM seemed to increase the fluorescent signal of the β2- and β5-subunits. However, the activation effect seems to be most pronounced at a concentration of 1 μM (FIG. 14A).

To further explore the role of the p38-pathway in proteasomal regulation, other known p38-inhibitors, PD98059, SB203580, SB202190, SKF86002 (1 and 5 μM) were incubated with MEL-JUSO cells. Interestingly, the compounds SB202190 and PD98059 both show a pronounced activation effect on the β2- and β5-subunits at a concentration of 5 μM (FIG. 14B). However, it must be taken into account that SB202190 inhibits both the p38α and p38β-isoforms [80, 83], while the compound SKF86002 inhibits only p38α. This suggests that both p38α and β-isoforms might be involved in regulating or enhancing proteasomal activity. On the other hand, PD98052, a MEK-1 inhibitor, also seems to enhance the activity of the β2- and β5-subunits; however, this might point out that other alternative MAPK pathways might be involved.

To quantify the enhancing effects of the compound PD169316, an in vivo activity assay using flow cytometry was performed. To this end KBM7 (chronic myeloma cell line lacking haploid karyotype except chromosome 8) cells were incubated with increasing PD169316 concentrations (0.01, 0.01, 0.1, 0.1, 1.0, 3.0, and 10.0 μM) for 16 h, stained with the fluorescent probe, and measured using flow cytometry. As in previous experiments, untreated cells served as a negative control, while cells incubated with MG132 served as a positive control. The plot of the concentration against the logarithmic fluorescence signal shows that the proteasomal activity increases with higher PD169116 concentrations (FIG. 14C). Thus, this assay confirms the activation effect of the β5-subunit of the proteasome observed in the SDS-PAGE gels. However, the fluorescent probe used to stain the cells for the flow-cytometry experiment, is only specific for the β5-subunit.

To further quantify the proteasomal activity after enhancement by the compound PD169316, a fluorogenic substrate conversion assay was used. The substrate Suc-LLVY-AMC is cleaved by the β5-subunit of the proteasome into the peptide and the fluorescent AMC (7-amino-4-methylcoumarin) group. To this end, KBM7-cells were incubated for 16 h with three different concentrations of PD169316 (1, 5 and 10 μM) as well as Mg132 (1 μM) as appositive control, and untreated cells (NT) as a negative control. After addition of fluorogenic substrate (100 μM), the assay was read out using a plate reader.

The results indicate that the compound PD169316 apparently enhances proteasomal activity, which is in agreement with the data gained from both the SDS-PAGE based assays and the flow cytometry experiment (FIG. 14D).

### Elucidation of Pathways

The compounds that were validated are believed to increase the proteasomal activity in cells. A series of experiments was conducted to further investigate the proteasome activating dynamics of these compounds.

It was previously determined that 16 hours of incubation with a proteasome activator leads to a strong increase in proteasome activity. But how this level of activation changed over time was not known. To test this, MelJuSo cells were incubated with 0.1, 0.5, 1.0 or 5.0 μM compound for 4, 16, 24 or 48 hours. This would allow us to monitor the effect of the compounds at different concentrations and time points and to evaluate if the effect is not only concentration but also time dependent. The results for the tested compounds are summarized in FIG. 15.

For most of the compounds the level of activation remains relatively constant over time. While higher concentrations of compound lead to a higher increase in FL-1 log 2 ratio, this does not change over time. While for some compounds such as Win 62,577 there is a mild decrease after 48 hours of incubation nothing suggests that any form of feedback mechanism is turned on to normalize proteasome activity. From this experiment it seems that proteasome activation remains stable as long as the compound is present. One exception to this general observation that activation is concentration but not time dependent is the compound DPCPX, as it displayed an increase in activation over time (FIG. 15). However further information is required in order to determine whether this is actually the case or the result of an experimental error.

Previous results suggested that the onset of maximal proteasome activation upon exposure with a compound was less than 6 hours. However the question remained how fast maximal activation is achieved and how long the activation remained after the compound is removed. To further investigate this, MelJuSo wild type cells were incubated with 5 μM Win 62,577 for one hour. Then the medium was changed for regular medium without the compound present. After one or two hours of “washout” cells were stained with the proteasome activity probe and analyzed with SDS-PAGE followed by fluorescent scanning of the gel. The results of this experiment are depicted in FIG. 17. After one hour of incubation with Win 62,577 a clear increase in signal is observed compared to the untreated control. After one hour of washout the signal is still more than the untreated control but less when compared to the no washout. After two hours of washout the signal detected is similar compared to the untreated control. From these results it appears both the onset and termination of increasing proteasome activity by this compound occurs in a timeframe of 1-2 hours. Taken together the results so far suggest that maximal activation is concentration dependent, occurs within two hours, is reversible and remains stable as long as the compound is present.

### Determining if Compounds Activate the Proteasome Directly or Indirectly

To determine if the compounds increase the 20S proteasome activity above basal levels directly or indirectly, the compounds were added to MelJuSo lysate and the conversion of fluorogenic substrates was measured as described. By first lysing cells the cellular environment is disrupted and signaling cascades or post-translational modifications no longer occur. If the compounds still activate the proteasome this must be via a direct interaction with the 20S proteasome. The results are depicted in FIG. 18. No significant change in AMC conversion was observed, suggesting that an intact cellular environment is required for the compounds to accomplish activation.

When this experiment was repeated with purified proteasome instead of MelJuSo lysate similar results were obtained (see FIG. 18). Taken together these data strongly suggest that activation of the proteasome by the compounds does not occur via a direct interaction.

### Protection of “Activators” Against MG132 Induced Cell Death

After it was established that the compound-induced increased proteasome activity is indirect, stable over time, occurs within 2 hours after addition and is reversible, the next question was if these compounds would convey any protection against cytotoxicity induced by a proteasome inhibitor. This was measured using a cell viability assay. In this assay the proteasome activity probe was not used as readout. Therefore compounds that influence the uptake of the probe, but do not activate the proteasome would have no effect in this assay. If the compounds would affect the uptake of reszurin, this would result in a increased conversion compared to untreated cells. Compounds that do have protective effect, but no increased uptake of resazurin, are strongly linked to the proteasome. To further exclude that the compounds influence the uptake of the proteasome activity probe or cellular uptake in general control experiments using a reduced version of the activity probe or carboxyfluorescein diacetate succinimidyl ester (CFDA-SE) was performed (data not shown).

To test this, cells were exposed for 24 hours to 1 μM of MG132 together with or without 5 μM of a proteasome activator and a cell survival assay was performed. After 8 hours of incubation with resazurin the fluorescent signal was measured and normalized to untreated cells. The results are depicted in FIG. 19A.

The first observation is that none of the proteasome activators at this concentration has an effect on cell survival by itself, while MG132 is highly cytotoxic after 24 hours. The second observation is that when both an activator and MG132 are present there is still a large decrease in cell viability but a protective effect is clearly visible. Next, cells were exposed to increasing concentrations of MG132 in the presence of 5 μM of proteasome activator. The resulting IC50 curves demonstrate a protective effect of the compounds against MG132 induced cytotoxicty (FIG. 19B). Furthermore there appears to be a correlation between the relative strength of proteasome activator and the magnitude of the protective effect. The data suggests that the presence of a proteasome activator delays MG132 induced cell death, rather than preventing it completely. At lower concentrations more cells survive when an activator is present. However at higher concentrations the cells die, despite the presence of an activator.

In summary: maximal increase of 20S and/or 26S proteasome activity induced by the identified proteasome activators occurs within 2 hours, is concentration dependent, reversible and stable as long as the compound is present. The presence of an activator delays against proteasome induced cytotoxicity. Furthermore the compounds appear to activate the 20S and/or 26S proteasome in an indirect way.

Having thus described in detail various embodiments of the present invention, it is to be understood that the invention defined by the above paragraphs is not to be limited to particular details set forth in the above description as many apparent variations thereof are possible without departing from the spirit or scope of the present invention.

### PSMB5 Expression Upon Exposure to Proteasome Activators

To investigate whether the effect of the previously identified proteasome activators was due to upregulation of transcription of proteasome subunits the expression of β5 subunits was determined by RT-PCR in Hela wild type cells after 16 hours exposure with 1 μM of activator.

### Methods:

mRNA expression levels of the proteasome subunit PSMB5 (β5), and the endogenous housekeeping gene β-glucuronidase (GUS) as a reference were quantified using real-time PCR analysis (SYBRgreen, Applied Biosciences) on a Chromo4 DNA Engine detection system (Biorad). Primers and concentrations used for the quantitative real-time PCR were as follows: PSMB5 forward (50 nM): CTTCAAGTTCCGCCATGGA; PSMB5 reverse (300 nM): CCGTCTGGGAGGCAA TGTAA; GUS forward (300 nM): GAAAATATGTGGTTG GAGAGCTCATT; GUS reverse (300 nM): CCGA GTGAAGATCCCCTTTTTA [85]. Real-time PCR was performed according to the manufacturer's instructions. Samples were amplified during 40 cycles of 15 s at 95° C. and 60 s at 60° C. Relative mRNA expression levels of the target genes in each sample were calculated using the comparative cycle time (Ct) method [86].

### REFERENCES

1. McNaught, K. S., et al., Failure of the ubiquitin-proteasome system in Parkinson's disease. Nat Rev Neurosci, 2001. 2(8): p. 589-94.
2. Schoenheimer, R., The Dynamic State of Body Constituents. 1942, Cambridge, Mass.: Harvard University Press.
3. Konstantinova, I. M., A. S. Tsimokha, and A. G. Mittenberg, Role of proteasomes in cellular regulation. Int Rev Cell Mol Biol, 2008. 267: p. 59-124.
4. Nickell, S., et al., Insights into the molecular architecture of the 26S proteasome. Proc Natl Acad Sci USA, 2009. 106(29): p. 11943-7.
5. Hanna, J. and D. Finley, A proteasome for all occasions. FEBS Lett, 2007. 581(15): p. 2854-61.
6. Sorokin, A V., E. R. Kim, and L. P. Ovchinnikov, Proteasome system of protein degradation and processing. Biochemistry (Mose), 2009. 74(13): p. 1411-42.
7. Bedford, L., et al., Assembly, structure, and function of the 26S proteasome. Trends Cell Biol, 2010. 20(7): p. 391-401.
8. Crews, C. M., Feeding the machine: mechanisms of proteasome-catalyzed degradation of ubiquitinated proteins. Curr Opin Chem Biol, 2003. 7(5): p. 534-9.
9. Orlowski, M., Selective activation of the 20 S proteasome (multicatalytic proteinase complex) by histone h3. Biochemistry, 2001. 40(50): p. 15318-26.
10. Dahlmann, B., et al., In vitro activation of the 20S proteasome. Enzyme Protein, 1993. 47(4-6): p. 274-84.
11. Kloetzel, P. M., A. Soza, and R. Stohwasser, The role of the proteasome system and the proteasome activator PA28 complex in the cellular immune response. Biol Chem, 1999. 380(3): p. 293-7.
12. Muchamuel, T., et al., A selective inhibitor of the immunoproteasome subunit LMP7 blocks cytokine production and attenuates progression of experimental arthritis. Nat Med, 2009. 15(7): p. 781-7.
13. Lecker, S. H., A. L. Goldberg, and W. E. Mitch, Protein degradation by the ubiquitin-proteasome pathway in normal and disease states. J Am Soc Nephrol, 2006. 17(7): p. 1807-19.
14. Berkers, C. R., et al., Profiling proteasome activity in tissue with fluorescent probes. Mol Pharm, 2007. 4(5): p. 739-48.
15. Kohler, A., et al., The axial channel of the proteasome core particle is gated by the Rpt2 ATPase and controls both substrate entry and product release. Mol Cell, 2001. 7(6): p. 1143-52.
16. Rabl, J., et al., Mechanism of gate opening in the 20S proteasome by the proteasomal ATPases. Mol Cell, 2008. 30(3): p. 360-8.
17. Saeki, Y. and K. Tanaka, Unlocking the proteasome door. Mol Cell, 2007. 27(6): p. 865-7.
18. Smith, D. M., et al., Docking of the proteasomal ATPases' carboxyl termini in the 20S proteasome's alpha ring opens the gate for substrate entry. Mol Cell, 2007. 27(5): p. 731-44.
19. Bech-Otsehir, D., et al., Polyubiquitin substrates allosterically activate their own degradation by the 26S proteasome. Nat Struct Mol Biol, 2009. 16(2): p. 219-25.
20, Ferrell, K., et al., Regulatory subunit interactions of the 26S proteasome, a complex problem. Trends Biochem Sci, 2000. 25(2): p. 83-8.
21. Koulich, E., X. Li, and G. N. DeMartino, Relative structural and functional roles of multiple deubiquitylating proteins associated with mammalian 26S proteasome. Mol Biol Cell, 2008. 19(3): p. 1072-82.
22. Navon, A. and A. Ciechanover, The 26 S proteasome: from basic mechanisms to drug targeting. J Biol Chem, 2009. 284(49): p. 11711-11718.
23. Marques, A. J., et al., Catalytic mechanism and assembly of the proteasome. Chem Rev, 2009. 109(4): p. 1509-36.
24. Kusmierczyk, A. R. and M. Hochstrasser, Some assembly required: dedicated chaperones in eukaryotic proteasome biogenesis. Biol Chem, 2008. 389(9): p. 1143-51.
25. Kusmierczyk, A. R., et al., A multimeric assembly factor controls the formation of alternative 20S proteasomes. Nat Struct Mol Biol, 2008. 15(3): p. 237-44.
26. Leggett, D. S., et al., Multiple associated proteins regulate proteasome structure and function. Mol Cell, 2002. 10(3): p. 495-507.
27. Satoh, K., et al., Assembly of the 26S proteasome is regulated by phosphorylation of the p45/Rpt6 ATPase subunit. Biochemistry, 2001. 40(2): p. 314-9.
28. Dohmen, R. J., I. Willers, and A. J. Marques, Biting the hand that feeds: Rpn4-dependent feedback regulation of proteasome function. Biochim Biophys Acta, 2007. 1773(11): p. 1599-604.
29. Huang, L. and C. H. Chen, Proteasome regulators: activators and inhibitors. Curr Med Chem, 2009. 16(8): p. 931-9.
30. Kuehn, L. and B. Dahlmann, Structural and functional properties of proteasome activator PA28. Mol Biol Rep, 1997. 24(1-2): p. 89-93.
31. Li, J. and M. Rechsteiner, Molecular dissection of the 11S REG (PA28) proteasome activators. Biochimie, 2001. 83(3-4): p. 373-83.
32. Murata, S., H. Yashiroda, and K. Tanaka, Molecular mechanisms of proteasome assembly. Nat Rev Mol Cell Biol, 2009. 10(2): p. 104-15.
33. Bailly, E. and S. I. Reed, Functional characterization of rpn3 uncovers a distinct 19S proteasomal subunit requirement for ubiquitin-dependent proteolysis of cell cycle regulatory proteins in budding yeast. Mol Cell Biol, 1999. 19(10): p. 6872-90.
34, Rosenzweig, R., et al., The central unit within the 19S regulatory particle of the proteasome. Nat Struct Mol Biol, 2008. 15(6): p. 573-80.
35. Li, D., et al., Structural basis for the assembly and gate closure mechanisms of the Mycobacterium tuberculosis 20S proteasome. EMBO J, 2010.
36. Glickman, M. H. and D. Raveh, Proteasome plasticity. FEBS Lett, 2005. 579(15): p. 3214-23.
37. Wang, H., et al., p45, an ATPase subunit of the 19S proteasome, targets the polyglutamine disease protein ataxin-3 to the proteasome. J Neurochem, 2007. 101(6): p. 1651-61.
38. Wang, X., et al., Proteomics of proteasome complexes and ubiquitinated proteins. Expert Rev Proteomics, 2007. 4(5): p. 649-65.
39. Hartmann-Petersen, R. and C. Gordon, Proteins interacting with the 26S proteasome. Cell Mol Life Sci, 2004. 61(13): p. 1589-95.
40. Spataro, V., C. Norbury, and A. L. Harris, The ubiquitin proteasome pathway in cancer. Br J Cancer, 1998. 77(3): p. 448-55.
41. Chumakov, P. M., Versatile functions of p53 protein in multicellular organisms. Biochemistry (Mosc), 2007. 72(13): p. 1399-421.
42. Sun, X. M., et al., Caspase activation inhibits proteasome function during apoptosis. Mol Cell, 2004. 14(1): p. 81-93.
43. Thiede, B., et al., Shotgun proteome analysis of protein cleavage in apoptotic cells. Proteomics, 2005. 5(8): p. 2123-30.
44. Aizawa, H., at al., Activation of the proteasome during Xenopus egg activation implies a link between proteasome activation and intracellular calcium release. Biochem Biophys Res Commun, 1996. 218(1): p. 224-8.
45. Dantuma, N. P., et al., A dynamic ubiquitin equilibrium couples proteasomal activity to chromatin remodeling. J Cell Biol, 2006. 173(1): p. 19-26.
46. Hanna, J., et al., A ubiquitin stress response induces altered proteasome composition. Cell, 2007. 129(4): p. 747-59.
47. Kurepa, J., et al., Proteasome regulation, plant growth and stress tolerance. Plant Signal Behav, 2009. 4(10): p. 924-7.
48. Demartino, G. N. and T. G. Gillette, Proteasomes: machines for all reasons. Cell, 2007. 129(4): p. 659-62.
49. Corn, P. G., Role of the ubiquitin proteasome system in renal cell carcinoma. BMC Biochem, 2007. 8 Suppl 1: p. S4.
50. Ciechanover, A. and P. Brundin, The ubiquitin proteasome system in neurodegenerative diseases: sometimes the chicken, sometimes the egg. Neuron, 2003. 40(2): p. 427-46.
51. Berkers, C. R., et al., Activity probe for in vivo profiling of the specificity of proteasome inhibitor bortezomib. Nat Methods, 2005. 2(5): p. 357-62.
52. Piva, R., et al., CEP-18770: A novel, orally active proteasome inhibitor with a tumor-selective pharmacologic profile competitive with bortezomib. Blood, 2008. 111(5): p. 2765-75.
53. Mateos, M. V. and J. F. San Miguel, Bortezomib in multiple myeloma. Best Pract Res Clin Haematol, 2007. 20(4): p. 701-15.
54. Berkers, C. A., et al., Comparison of the specificity and activity profiles of the proteasome inhibitors bortezomib and CEP-18770.
55. Zhao, X. and J. Yang, Amyloid-beta Peptide Is a Substrate of the Human 20S Proteasome. ACS Chemical Neuroscience, 2010.
56. Lee, B. H., et al., Enhancement of proteasome activity by a small-molecule inhibitor of USP14. Nature, 2010. 467(7312): p. 179-84.
57. Tanaka, K, et al., A high molecular weight protease in the cytosol of rat liver. I. Purification, enzymological properties, and tissue distribution. J Biol Chem, 1986. 261(32): p. 15197-203.
58. Dahlmann, B., et al., Activation of the multicatalytic proteinase from rat skeletal muscle by fatty acids or sodium dodecyl sulphate. Biochem J, 1985. 228(1): p. 171-7.
59. Watanabe, N. and S. Yamada, Activation of 20S proteasomes from spinach leaves by fatty acids. Plant Cell Physiol, 1996. 37(2): p. 147-51.
60. Wilk, S, and W. E. Chen, Synthetic peptide-based activators of the proteasome. Mol Biol Rep, 1997. 24(1-2): p. 119-24.
61. Ohkubo, I., et al., Human erythrocyte multicatalytic proteinase: activation and binding to sulfated galacto-and lactosylceramides. Biochem Biophys Res Commun, 1991. 174(3): p. 1133-40.
62. Matsumura, K. and K. Aketa, Proteasome (multicatalytic proteinase) of sea urchin sperm and its possible participation in the acrosome reaction. Mol Reprod Dev, 1991. 29(2): p. 189-99.
63. Ruiz de Mena, I., et al., Kinetic mechanism of activation by cardiolipin (diphosphatidylglycerol) of the rat liver multicatalytic proteinase. Biochem J, 1993. 296 Pt 1): p. 93-7.
64. Huang, L., P. Ho, and C. H. Chen, Activation and inhibition of the proteasome by betulinic acid and its derivatives. FEBS Lett, 2007. 581(25): p. 4955-9.
65. Patterson, C. and D. M. Cyr, eds. Ubiquitin-Proteasome Protocols. Methods in Molecular Biology, ed. J. M. Walker. Vol. 301. 2005, Humana Press: Totowa, N. J. 381.
66. Laporte, D., et al., Reversible cytoplasmic localization of the proteasome in quiescent yeast cells. J Cell Biol, 2008. 181(5): p. 737-45.
67. Carette, J. E., et al., Haploid genetic screens in human cells identify host factors used by pathogens. Science, 2009. 326(5957): p. 1231-5.
68. Krzywda, S., et al., Crystallization of gankyrin, an oncoprotein that interacts with CDK4 and the S6b (rpt3) ATPase of the 19S regulator of the 26S proteasome. Acta Crystallogr D Biol Crystallogr, 2003. 59(Pt 7): p. 1294-5.
69. Higashitsuji, H., et al., The oncoprotein gankyrin negatively regulates both p53 and RB by enhancing proteasomal degradation. Cell Cycle, 2005. 4(10): p. 1335-7.
70. Gillette, T. G., et al., Differential roles of the COOH termini of AAA subunits of PA700 (19 S regulator) in asymmetric assembly and activation of the 26 S proteasome. J Biol Chem, 2008. 283(46): p. 31813-22.
71. Husnjak, K., et al., Proteasome subunit Rpn13 is a novel ubiquitin receptor. Nature, 2008. 453(7194): p. 481-8.
72. Babbitt, S. E., et al., ATP hydrolysis-dependent disassembly of the 26S proteasome is part of the catalytic cycle. Cell, 2005. 121(4): p. 553-65.
73. Chen, C., et al., Subunit-subunit interactions in the human 26S proteasome. Proteomics, 2008. 8(3): p. 508-20.
74. Chong, C R., et al., A clinical drug library screen identifies astemizole as an antimalarial agent. Nature Chemical Biology, 2006. 2: p. 415-416.
75. Weissman, A. M., Shabek, N., Chiechanover, A., The predator becomes the prey: regulating the ubiquitin system by ubiquitylation and degradation. Nature Reviews, 2001, 12, 605-621.
76. Finley, D., Recognition and Processing of Ubiquitin-Protein Conjugates by the Proteasome, Annu. Rev. Biochem., 2009, 78, 477-513.
77. Hedge, A. N., The ubiquitin-proteasome pathway and synaptic plasticity, Learning and Memory, 2010, 17, 314-327.
78. Voges, D. Zwickl, Baumeister, W., The 26S proteasome: A molecular machine designed for controlled proteolysis. Annu. Rev. Biochem., 1999, 68, 1015-68.
79. Glickmann, M. H., Chiechanover, A., The Ubiquitin-Proteasome Proteolytic Pathway: Destruction for the sake of Construction, Physiol. Rev., 2001, 82, 373-428.
80. Ruschak, A. M., et al., Novel Proteasome Inhibitors to Overcome Bortezomib Resistance, J. Natl. Cancer Inst., 2011, 103813), 1007-17.
81. Stadtmueller, B., Hill, C. P., Proteasome Activators, Molecular Cell, 2011, 41, 8-19.
82. Li, X., Wood, T. E., Sprangers, R., et al. Effect of Noncompetitive Proteasome Inhibition on Bortezomib Resistance, Oxford Journals, 2010, 102(14), 1069-1092.
83. Hotokezaka, H., Sakai, E., Kanaoka, K., et al., U0126 and PD98059, Specific Inhibitors of MEK, Accelerate Differentiation of RAW 264.7 Cells into Osteoclast-like Cells, J Biol Chem, 2002, 277(49), 47366-72.
84. De Jong, A., Schuurmann, K. G., Rodenko, B., et al., Fluorescence-Based Proteasome Activity Profiling, Chemical Proteomics: Methods and Protocols, Methods in Molecular Biology, Vol., 803, Chapter 13, 183-204.
85. Oerlemans, R. et al. Molecular basis of bortezomib resistance: proteasome subunit beta5 (PSMB5) gene mutation and overexpression of PSMB5 protein. Blood 112, 2489-2499 (2008).
86. Meijerink, J. et al. A novel method to compensate for different amplification efficiencies between patient DNA samples in quantitative real-time PCR. J. Mol. Diagn. 3, 55-61 (2001).

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A composition for increasing an activity of 20S and/or 26S proteasome above basal levels, comprising a compound, which is an activator of the 20S and/or 26S proteasome, selected from the group consisting of calcium channel modulators, cAMP inhibitors, antiandrogens, methylbenzonium salts, PD 169316 and proflavine and a pharmaceutically acceptable carrier.

2. The composition of claim 1, in which the compound is selected from the group consisting of methylbenzethonium, PD 169316, proflavine, cyclosporin A, loperamide, metergoline, pimozide, Win 62,577, verapamil, cyproterone, dipyrimadole, DPCPX, fenofibrate, medroxyprogesterone, mifepristone, pimozide, cyproterone, mifepristone, medroxyprogesterone and structural analogs thereof.

3. A method of increasing an activity of the 20S and/or 26S proteasome above basal levels administering a therapeutically effective amount of a compound that is an activator comprising of 20S and/or 26S proteasome to a patient in need thereof.

4. The method of claim 3, in which the compound directly activates direct activation of the 20S and/or 26S proteasome.

5. The method of claim 3, in which the compound indirectly activates the 20S and/or 26S proteasome.

6. The method of claim 3, wherein the patient in need thereof is suffering from a neurodegenerative disease/disorder, a disease characterized by protein aggregation and/or protein deposition, an autoimmune disease, an infectious disease, cancer or inflammation.

7. The method of claim 6, wherein:
the neurodegenerative disease/disorder, disease characterized by protein aggregation and/or protein deposition is selected from the group consisting of Alzheimer's disease, Parkinson's disease, amyotrophic lateral sclerosis (ALS), Huntington's disease, transmissible spongiform encephalopaties (TSEs), Creutzfeld-Jakob disease, systemic amyloidosis, prion based diseases and diseases caused by polyglutamine repeats;
the autoimmune disease is selected from the group consisting of alopecia areata, ankylosing spondylitis, arthritis, antiphospholipid syndrome, autoimmune Addison's disease, autoimmune hemolytic anemia, autoimmune inner ear disease (also known as Meniers disease), autoimmune lymphoproliferative syndrome (ALPS), autoimmune thrombocytopenic purpura, autoimmune hemolytic anemia, autoimmune hepatitis, Bechet's disease, Crohn's disease, diabetes mellitus type 1, glomerulonephritis, Graves' disease, Guillain-Barre syndrome, inflammatory bowel disease, lupus nephritis, multiple sclerosis, myasthenis gravis, pemphigus, pemicous anemia, polyarteritis nodosa, polymyositis, primary billiary cirrhosis, psoriasis, Raynaud's Phenomenon, rheumatic fever, rheumatoid arthritis, scleroderma, Sjogren's syndrome, systemic lupus erythematosus (SLE), ulcerative colitis, vitiligo, and Wegener's granulamatosis;
the infectious disease is selected from the group consisting of disease associated with defective antigen presentation via MHC molecules;
the cancer is selected from the group consisting of leukemia; carcinoma of bladder, breast, colon, kidney, liver, lung, ovary, pancreas, stomach, cervix, thyroid, prostate, head, neck and skin; hematopoietic tumors of lymphoid lineage, acute lyphocytic leukemia; B-cell lymphoma; Burkett's lymphoma; hematopoietic tumors of myeloid lineage, acute and chronic myelogenous leukemias and promyelocytic leukemia; tumors of mesenchymal origin, fibrosarcoma, rhabdomyasarcoma; melanoma; seminoma; teratocarcinoma; osteosarcoma; neuroblastoma and glioma; and
the inflammation is selected from the group consisting of rheumatoid arthritis, spondyloathopathies, gouty arthritis, osteoarthritis, systemic lupus erythematosis, juvenile arthritis, bronchitis, bursitis, gastritis, inflammatory bowel disease, ulcerative colitis, acne vulgaris, asthma, autoimmune dieases, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

8. The method of claim 6, wherein the patient is suffering from Alzheimer's disease, Parkinson's disease or a prion-induced disease.

9. A method of identifying a compound, which increases an activity of 20S and/or 26S proteasome above basal levels, comprising:
i. obtaining a compound (a) from a compound library or other source;
ii. obtaining a cell type which expresses constitutive proteasome and incubating the cell type with the compound (a);
iii. adding a fluorescent probe to a cell culture of the cell type, which binds covalently to a catalytic subunit of a 20S or a 26S proteasome to transform the catalytic subunit into a 20S or 26S proteasome-fluorescent probe complex;
iv. measuring fluorescence (FL-1) of the 20S or 26S proteasome-fluorescent probe to create a score for proteasome activity and converting the score to an FL-1 log 2 ratio relative to an average of untreated cells.
v. identifying a compound (a) which has a FL-1 log 2 ratio greater than 1.00;
vi. validating the identified compound (a) in step v. by:
a. optionally repeating steps i.-iv.; and
b. repeating steps i.-iii., followed by lysing the cells to form a cell lysate which is resolved by SDS-PAGE and subsequently analyzed by fluorescent scanning of the resulting gel;
vii. identifying a compound (a) which still has a FL-1 log 2 ratio greater than 1.00 after optional step vi. a. and has bands that show dose-dependent increased fluorescence for the β2 and β5 subunits of the 20S or 26S proteasome after step vi. b.

10. A pharmaceutical composition comprising one or more compounds identified as being activators of a 20S and/or 26S proteasome by the process of claim 9.

11. The method of claim 9, wherein compound (a):
i. has a side scatter (SSC) less than average of DMSO+3× Standard Deviation (SD); and/or has a number of events greater than DMSO−3×SD; and
ii. has FL-2 and/or FL-3 less than average of DMSO+3×SD.

12. A method of increasing an activity of a 20S and/or 26S proteasome above basal levels by administering a therapeutically effective amount of a compound identified by the method of claim 9 to a patient in need thereof.

13. The method of claim 12, wherein the method of increasing the activity is via direct activation of the 20S and/or 26S proteasome.

14. The method of claim 12, wherein the method of increasing the activity is via indirect activation of the 20S and/or 26S proteasome.

15. The method of claim 12, wherein the patient in need thereof is suffering from a neurodegenerative disease/disorder, a disease characterized by protein aggregation and/or protein deposition, an autoimmune disease, an infectious disease, cancer or inflammation.

16. The method of claim 15, wherein:
the neurodegenerative disease/disorder, disease characterized by protein aggregation and/or protein deposition is selected from the group consisting of Alzheimer's disease, Parkinson's disease, amyotrophic lateral sclerosis (ALS), Huntington's disease, transmissible spongiform encephalopaties (TSEs), Creutzfeld-Jakob disease, systemic amyloidosis, prion based diseases and diseases caused by polyglutamine repeats;
the autoimmune disease is selected from the group consisting of alopecia areata, ankylosing spondylitis, arthritis, antiphospholipid syndrome, autoimmune Addison's disease, autoimmune hemolytic anemia, autoimmune inner ear disease (also known as Meniers disease), autoimmune lymphoproliferative syndrome (ALPS), autoimmune thrombocytopenic purpura, autoimmune hemolytic anemia, autoimmune hepatitis, Bechet's disease, Crohn's disease, diabetes mellitus type 1, glomerulonephritis, Graves' disease, Guillain-Barre syndrome, inflammatory bowel disease, lupus nephritis, multiple sclerosis, myasthenis gravis, pemphigus, pemicous anemia, polyarteritis nodosa, polymyositis, primary billiary cirrhosis, psoriasis, Raynaud's Phenomenon, rheumatic fever, rheumatoid arthritis, scleroderma, Sjogren's syndrome, systemic lupus erythematosus (SLE), ulcerative colitis, vitiligo, and Wegener's granulamatosis;
the infectious disease is selected from the group consisting of disease associated with defective antigen presentation via MHC molecules;
the cancer is selected from the group consisting of leukemia; carcinoma of bladder, breast, colon, kidney, liver, lung, ovary, pancreas, stomach, cervix, thyroid, prostate, head, neck and skin; hematopoietic tumors of lymphoid lineage, acute lyphocytic leukemia; B-cell lymphoma; Burkett's lymphoma; hematopoietic tumors of myeloid lineage, acute and chronic myelogenous leukemias and promyelocytic leukemia; tumors of mesenchymal origin, fibrosarcoma, rhabdomyasarcoma; melanoma; seminoma; teratocarcinoma; osteosarcoma; neuroblastoma and glioma; and
the inflammation is selected from the group consisting of rheumatoid arthritis, spondyloathopathies, gouty arthritis, osteoarthritis, systemic lupus erythematosis, juvenile arthritis, bronchitis, bursitis, gastritis, inflammatory bowel disease, ulcerative colitis, acne vulgaris, asthma, autoimmune dieases, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

17. The method of claim 15, wherein the patient is suffering from Alzheimer's disease, Parkinson's disease or a prion-induced disease.

18. A compound of formula I: 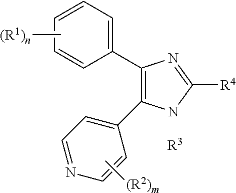
wherein,
R¹ is halogen, hydroxyl, amino, or C₁-C₄ alkyl;
R² is halogen, hydroxyl, amino, or C₁-C₄ alkyl;
R³ is hydrogen or C₁-C₄ alkyl;
R⁴ is phenyl or phenyl substituted with halogen, hydroxyl, amino, C₁-C₄ alkyl, nitro, sulfinyl C₁-C₄ alkylsulfinyl; sulfonyl or C₁-C₄ alkylsulfonyl; or
R³ and R⁴ together form a 5- or 6-membered ring with at least one additional heteroatom selected from the group consisting of O, N and S;
n is 0-5; and
m is 0-4.

19. The compound of claim 18, wherein
R¹ is halogen;
R² is halogen;
R³ is hydrogen or CH₃;
R⁴ is phenyl substituted with halogen, hydroxyl, amino, C₁-C₄ alkyl, nitro, methylsulfinyl or methylsulfonyl; or
R³ and R⁴ together form a 5-6 membered ring with at least one additional heteroatom selected from the group consisting of O, N and S;
n is 0-2; and
m is 0-1.

20. The compound of claim 18, wherein
R¹ is F;
R³ is hydrogen or CH₃;
R⁴ is phenyl substituted with F, hydroxyl, amino, CH₃, nitro, or methylsulfinyl; or
R³ and R⁴ together form a 5 membered ring with S as one additional heteroatom;
n is 0-1; and
m is 0.

21. The compound of claim 18, wherein the compound is [chemical structure]


SEQUENCE LISTING (7 sequences)

SEQ ID NO: 1 - protein, 6 aa, Artificial Sequence
  Description of Artificial Sequence Synthetic peptide
XXXLLL

SEQ ID NO: 2 - protein, 4 aa, Artificial Sequence
  Description of Artificial Sequence Synthetic peptide
LLVY

SEQ ID NO: 3 - protein, 4 aa, Artificial Sequence
  Description of Artificial Sequence Synthetic peptide
LLVY

SEQ ID NO: 4 - DNA, 19 bp, Artificial Sequence
  Description of Artificial Sequence Synthetic primer
cttcaagttccgccatgga

SEQ ID NO: 5 - DNA, 20 bp, Artificial Sequence
  Description of Artificial Sequence Synthetic primer
ccgtctgggaggcaatgtaa

SEQ ID NO: 6 - DNA, 26 bp, Artificial Sequence
  Description of Artificial Sequence Synthetic primer
gaaaatatgtggttggagagctcatt

SEQ ID NO: 7 - DNA, 22 bp, Artificial Sequence
  Description of Artificial Sequence Synthetic primer
ccgagtgaagatccccttttta